(19) United States
(12) Patent Application Publication
(10) Pub. No.: US 2023/0399377 A1
(43) Pub. Date: Dec. 14, 2023

(54) INHIBITORS OF ANGIOGENIC FACTORS

(71) Applicant: ELUMINEX BIOSCIENCES (SUZHOU) LIMITED, Suzhou, Jiangsu (CN)

(72) Inventors: Jinzhong ZHANG, Orinda, CA (US); Wei Yong SHEN, Carlton, New South Wales (AU); Charles SEMBA, Palo Alto, CA (US); John Zhenze HU, Pittsford, NY (US); Zhen Qin XIA, Albany, CA (US)

(21) Appl. No.: 18/250,977

(22) PCT Filed: Oct. 26, 2021
(86) PCT No.: PCT/CN2021/126470

§ 371 Date: Apr. 27, 2023

Related U.S. Application Data
(60) Provisional application No. 63/204,904, filed on Oct. 30, 2020.

(51) Int. Cl. C07K 14/71

(52) U.S. Cl. CPC C07K 14/71; C07K 2319/30; A61K 38/00

(57) ABSTRACT

A recombinant human Fc fusion protein comprises an Ig-like domain of a first human VEGF receptor, another different Ig-like domain of a second human VEGF receptor, at least yet another Ig-like domain of a third human VEGF receptor, and the Fc portion of the human IgG1. The fusion protein is useful in treating or controlling an ocular disease, condition, or disorder that has etiology in aberrant angiogenesis.

### BACKGROUND OF THE INVENTION

The present invention relates to inhibitors of angiogenic factors. In particular, the present invention relates to inhibitors of members of the family of vascular endothelial growth factors (“VEGF family members”). More particular, the present invention relates to inhibitors of activity of VEGF family members, uses, and processes for production of such inhibitors.

The major cellular components of the mammalian vascular system are the endothelium, smooth muscle cells, and pericytes. Endothelial cells form the lining of the inner surface of all blood vessels in the mammal and constitute a non-thrombogenic interface between blood and tissue. Therefore, the proliferation of endothelial cells is an important component for the development of new capillaries and blood vessels which, in turn, is a necessary process for the growth and/or regeneration of mammalian tissues.

A family of secreted polypeptides has been shown to play an extremely important role in promoting endothelial cell proliferation and angiogenesis. A pathological feature of uncontrolled angiogenesis caused by VEGF over-expression is increased vascular permeability, which results in fluid leakage into, and swelling of, the surrounding tissues. In mammals, this family consists of five related growth factors having highly conserved receptor-binding structure: vascular endothelial growth factors A-D (“VEGF-A,” “VEGF-B,” “VEGF-C,” and “VEGF-D”) and placental growth factor (“PGF”). In this disclosure, this family of growth factors is also referred to as the VEGF family. These growth factors act through a family of cognate receptor tyrosine kinases, which exist only on the surface of vascular endothelial cells, to stimulate formation of blood vessels: VEGF receptor-1 (“VEGFR-1,” also known as “fit-1”), VEGF receptor-2 (“VEGFR-2,” also known as “KDR” in humans and “flk-1” in mice), VEGF receptor-3 (“VEGFR-3,” also known as “flt-4”).

VEGF-A (also sometimes simply referred to as VEGF) has emerged as the most important member of this family of growth factors. Human VEGF-A is expressed in a variety of tissues as multiple homodimeric forms (121, 145, 165, 183, 189 and 206 amino acids per monomer), wherein each form arises as a result of alternative splicing of a single RNA transcript.

Since VEGFs promote vascular endothelial cell proliferation and angiogenesis, they may be useful for the therapeutic treatment of numerous conditions in which a growth-promoting activity on the vascular endothelial cells is beneficially important; for example, in treatment of ulcers, vascular injuries, and myocardial infarction.

In contrast, however, while vascular endothelial proliferation is desirable under certain circumstances, vascular endothelial proliferation and angiogenesis are also undesirable components of a variety of diseases and disorders including tumor growth and metastasis, rheumatoid arthritis, psoriasis, atherosclerosis, diabetic retinopathy, retrolental fibroplasia, neovascular glaucoma, neovascular age-related macular degeneration, hemangiomas, immune rejection of transplanted corneal tissue and other tissues, and chronic inflammation. In individuals suffering from any of these disorders, one would want to inhibit, or at least substantially reduce, the endothelial proliferating activity of VEGFs.

Each of fit-I, KDR, and flt-4 tyrosine kinase receptors has seven extracellular immunoglobulin-like (“Ig-like”) domains that are available for ligand binding, a transmembrane domain that serves to anchor the receptor on the surface of cells in which it is expressed and an intracellular catalytic tyrosine kinase domain. Flt-1 binds VEGF-A, VEGF-B, and P1GF. KDR binds VEGF-A, VEGF-C, and VEGF-D. Flt-4 binds VEGF-C and VEGF-D.

In view of the role of the growth factors of the VEGF family in vascular endothelial proliferation and angiogenesis, and the role that these processes play in many different diseases and disorders, it is desirable to have a pharmacological means for reducing or inhibiting one or more of the biological activities of these growth factors in patients whose pathological conditions are rooted in aberrant angiogenesis. It is also desirable to have a pharmacological means for improved treatment or control of pathological conditions that are rooted in aberrant angiogenesis.

### SUMMARY OF THE INVENTION

As used herein, the term “control” also includes reduction, alleviation, amelioration, or prevention.

In general, the present invention provides fusion or chimeric polypeptides or proteins, methods of producing and compositions comprising the same, and methods for treating or controlling at least a pathological condition in a subject, which condition has etiology in aberrant angiogenesis.

In one aspect, the present invention provides fusion or chimeric polypeptides that are capable of binding substantially to one or more VEGF family members; thereby, reducing or inhibiting their binding to VEGF receptors.

In another aspect, a fusion or chimeric polypeptide of the present invention comprises an Ig-like domain of a first VEGF receptor, an Ig-like domain of a second VEGF receptor, and at least an Ig-like domain of a third VEGF receptor.

In still another aspect, a fusion or chimeric polypeptide of the present invention comprises: (a) Ig-like domain 2, or substantially Ig-like domain 2, of human VEGFR-1 (or fit-1); (b) Ig-like domain 3, or substantially Ig-like domain 3, of human VEGFR-2 (or KDR); and (c) Ig-like domains 1 and 2, or substantially Ig-like domains 1 and 2, of human VEGFR-3 (or fit-4).

In still another aspect, a fusion or chimeric polypeptide of the present invention comprises: (a) Ig-like domain 2, or substantially Ig-like domain 2, of human VEGFR-1; (b) Ig-like domain 3, or substantially Ig-like domain 3, of human VEGFR-2; and (c) Ig-like domains 1, 2, and 3, or substantially Ig-like domains 1, 2, and 3, of human VEGFR-3.

In still another aspect, the human VEGFR-3 is the wild-type human VEGFR-3 having the amino acid sequence listed in SEQ ID NO: 13; and the Ig-like domain 1 of human VEGFR-3 that is included in any fusion protein of the present invention has the amino acid sequence listed in SEQ ID NO: 14. A fusion protein of the present invention comprises Ig-like domain 1, or substantially Ig-like domain 1, of human VEGFR-3; wherein the amino acid sequence at positions 80-82 of SEQ ID: 14 is NDT, NDS, NXT, or NXS; and X is any amino acid. In one embodiment, X is a naturally occurring amino acid.

In yet another aspect, the fusion or chimeric polypeptide of the present invention comprises: (a) Ig-like domain 2, or substantially Ig-like domain 2, of human VEGFR-1; (b) Ig-like domain 3, or substantially Ig-like domain 3, of human VEGFR-2; and (c) Ig-like domains 1 and 2, or substantially Ig-like domains 1 and 2, of human VEGFR-3; wherein the amino acid sequence of human VEGFR-3 is listed in SEQ ID NO: 13; and wherein the amino acid sequence at positions 104-106 is NDT, NDS, NXT, or NXS; wherein X is any amino acid. In one embodiment, X is a naturally occurring amino acid.

In yet another aspect, the fusion or chimeric polypeptide of the present invention comprises: (a) Ig-like domain 2, or substantially Ig-like domain 2, of human VEGFR-1 (or fit-1); (b) Ig-like domain 3, or substantially Ig-like domain 3, of human VEGFR-2 (or KDR); and (c) Ig-like domains 1 and 2, or substantially Ig-like domains 1 and 2, of human VEGFR-3 (or fit-4); linked to a multimerizing component.

In one embodiment, said multimerizing component comprises a portion of an Fc domain of human IgG1.

In another embodiment, said multimerizing component comprises substantially the last two heavy-chain domains at the amino terminus of IgG1; in other words, Fc CH2 and CH3 domains of human IgG1.

In still another aspect, the present invention provides an isolated nucleic acid molecule encoding said fusion or chimeric polypeptide.

In still another aspect, the present invention provides a vector that comprises said nucleic acid molecule, including an expression vector comprising said nucleic molecule operatively linked to an expression control sequence.

In still another aspect, the present invention provides a host-vector system for the production of said fusion or chimeric polypeptide that comprises the expression vector in a suitable host cell.

In another aspect, the present invention provides a method of producing a fusion or chimeric polypeptide, which method comprises: (a) growing cells of the host-vector system under conditions permitting production of the fusion or chimeric protein; and (b) recovering the fusion or chimeric polypeptide so produced.

In still another aspect, the present invention provides a method for treating or controlling at least a disease, condition, or disorder, in a subject, which has etiology in aberrant angiogenesis.

In yet another aspect, the present invention provides a method for treating or controlling at least an ocular disease, condition, or disorder, in a subject, which has etiology in aberrant angiogenesis.

Other features and advantages of the present invention will become apparent from the following detailed description and claims.

### BRIEF DESCRIPTION OF THE DRAWINGS

FIG. 1 shows schematically a fusion polypeptide of the present invention, comprising Ig-like domain 2 of VEGFR-1, Ig-like domain 3 of VEGFR-2, Ig-like domains 1 and 2 of VEGFR-3, and an Fc domain.

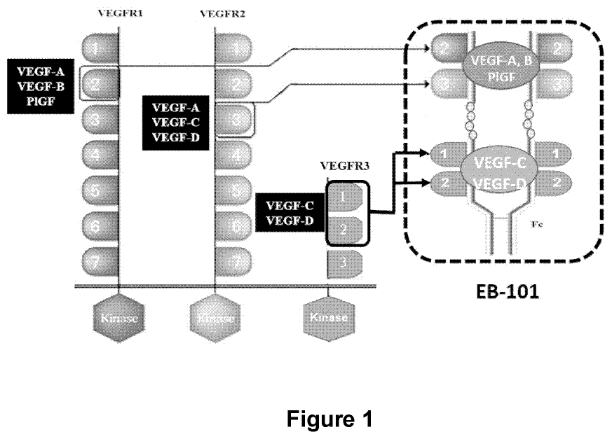

FIG. 2 shows the SDS-PAGE and SEC-HPLC results for the transient expression of EB-101 in HEK293 cells.

FIG. 3 shows the SDS-PAGE and SEC-HPLC results for the transient expression of EB-101 in CHO cells.

FIG. 4 shows the comparison of the binding affinity of EB-101 with aflibercept for human recombinant human VEGF-A₁₆₅ using SPR Biacore.

FIG. 5 shows the comparison of the binding affinity of EB-101 with aflibercept for human VEGF-B 167/189 using SPR Biacore.

FIG. 6 shows the comparison of the binding affinity of EB-101 with aflibercept for human VEGF-C using SPR Biacore.

FIG. 7 shows the comparison of the binding affinity of EB-101 with aflibercept for human VEGF-D using SPR Biacore.

FIG. 8 shows the comparison of the binding affinity of EB-101 with aflibercept for human P1GF using SPR Biacore.

FIG. 9 shows the comparison of the effect of EB-101 with aflibercept and bevacizumab on inhibition of VEGF-A₁₆₅ mediated VEGFR-2 signaling in VEGFR-2-NFAT-RE luciferase reporter cells.

FIG. 10 shows the comparison of the effect of EB-101 with aflibercept on inhibition of VEGF-C mediated VEGFR-2 signaling in VEGFR-2-NFAT-RE luciferase reporter cells.

FIG. 11 shows the comparison of EB-101 with aflibercept and a recombinant human VEGFR-3 containing 7 extracellular domains (rhVEGFR-3-7ECDs) on their inhibitory effect on VEGF-C mediated VEGFR-2 signaling in VEGFR-2-NFAT-RE luciferase reporter cells.

FIG. 12 shows the quantitative analyses of the effect of EB-101 with aflibercept and a recombinant human VEGFR-3 containing 7 extracellular domains (rhVEGFR-3) on inhibition of VEGFR-2 signaling in VEGFR-2-NFAT-RE luciferase reporter cells stimulated by VEGF-A₁₆₅ and VEGF-C.

FIG. 13 shows the comparison of the effect of EB-101 with aflibercept and bevacizumab on VEGF-A₁₆₅-mediated proliferation of human lymphatic endothelial cells (HLECs).

FIG. 14 shows the comparison of the effect of EB-101 with aflibercept and a recombinant human VEGFR3 containing 7 extracellular domains (rhVEGFR3 ECDs) on VEGF-C mediated HLEC proliferation.

FIG. 15 shows the quantitative analyses of EB-101, aflibercept and a recombinant human VEGFR3 containing 7 extracellular domains (rhVEGFR3 ECDs), on VEGF-A₁₆₅ plus VEGF-C mediated HLEC proliferation.

FIG. 16 shows schematically a fusion polypeptide of the present invention, comprising Ig-like domain 2 of VEGFR-1, Ig-like domain 3 of VEGFR-2, Ig-like domains 1, 2, and 3 of VEGFR-3, and an Fc domain.

FIG. 17 shows the comparison of the effect of EB-101BIb with aflibercept, EB-101, EB-101BIa on VEGF-A mediated VEGFR-2 signaling in VEGFR-2-NFAT-RE luciferase reporter cells.

FIG. 18 shows the comparison of the effect of EB-101BIb with aflibercept, EB-101 and EB-101BIa on VEGFR-2 signaling in VEGFR-2-NFAT-RE luciferase reporter cells stimulated by recombinant human VEGF-C and -D.

FIG. 19 shows the comparison of the inhibitory effect of EB-101BIb with aflibercept, EB-101 and a combination of aflibercept with a recombinant human VEGFR-3 on VEGF-A, -C, and -D mediated VEGFR-2 signaling in VEGFR-2-NFAT-RE luciferase reporter cells.

FIG. 20 shows the comparison of the effect of EB-101BIb with aflibercept and EB-101BIa on VEGF-A₁₆₅-mediated proliferation of human lymphatic endothelial cells (HLECs).

FIG. 21 shows the comparison of the effect of EB-101BIb with aflibercept, EB-101BIa and a recombinant human VEGFR-3 containing 7 extracellular domains on VEGF-C and VEGF-D mediated HLEC proliferation.

FIG. 22 shows the comparison of the effect of EB-101BIb with aflibercept, a recombinant human VEGFR-3 containing 7 extracellular domains (7 ECDs) and a combination of aflibercept with the VEGFR-3 on VEGF-A, —C and -D mediated human lymphatic endothelial cell (HLEC) proliferation.

### DETAILED DESCRIPTION OF THE INVENTION

In the present disclosure, the terms “fusion polypeptide,” “fusion protein,” “chimeric polypeptide,” and “chimeric protein” are used interchangeably.

The present invention provides a binding construct that is capable of binding at least one member of the VEGF family (“VEGF family member”); thereby, rendering said VEGF family member substantially unavailable for binding to a VEGF receptor on endothelial cells. As a result, a binding construct of the present invention substantially inhibits biological activity of said VEGF family member in promoting angiogenesis; thereby, controlling a pathological condition having etiology in aberrant angiogenesis.

The binding construct of the present invention comprises or consists of a fusion or chimeric polypeptide or protein and comprises one or more binding units that are associated with each other by covalent or other forms of attachment. A binding construct of the present invention is capable of binding a VEGF family member or a portion thereof and does so with high affinity. A binding unit preferably comprises at least one peptide or polypeptide. While a binding unit preferably comprises a single polypeptide, it may comprise multiple polypeptides if a single polypeptide is not sufficient for binding a particular growth factor. When more than one binding unit or polypeptide segment is in a given binding construct, the binding units may be joined together directly or through a linker. A binding construct may further include a heterologous peptide or other chemical moieties. Such additions can modify binding construct properties such as stability, solubility, toxicity, serum half-life, immunogenicity, detectability, or other properties.

The term “high affinity” is used in a physiological context pertaining to the relative affinity of the binding construct for the VEGF family members in vivo in a mammal, such as a laboratory test animal, a domesticated farm or pet animal, or a human. The targeted VEGF members in the present invention have characteristic affinities for their receptors in vivo, typically measured in terms of sub-nanomolar dissociation constants (K_{d}). For the purposes of this invention, a binding construct of the present invention can bind to its target VEGF family member(s) with a K_{d} less than or equal to about 1, or about 5, or about 10, or about 50, or about 100, or about 500, or about 1000 times the K_{d} of the natural growth factor-receptor pair.

In one aspect, a fusion or chimeric polypeptide or protein of the present invention comprises a first polypeptide binding unit, a second polypeptide binding unit, and a third polypeptide binding unit, operatively linked together. A polypeptide binding unit may be referred to herein in an abbreviated way as “a binding unit.” In general, the first, second, and third binding units may be linked together in any order, directly or through an intervening linker.

In another aspect, a targeted VEGF family member may bind to one or more binding units of the fusion or chimeric polypeptide.

In still another aspect, a binding unit comprises substantially an Ig-like domain of a VEGF receptor.

In another aspect, a fusion or chimeric polypeptide of the present invention comprises a first binding unit that comprises an Ig-like domain of a first VEGF receptor; a second binding unit that comprises an Ig-like domain of a second VEGF receptor; and a third binding unit that comprises at least an Ig-like domain of a third VEGF receptor.

In another aspect, the fusion or chimeric polypeptide of the present invention comprises an Ig-like domain of a first VEGF receptor, an Ig-like domain of a second VEGF receptor, and at least an Ig-like domain of a third VEGF receptor; wherein said third VEGF receptor comprises human VEGFR-3 (or fit-4) having an amino acid sequence listed in SEQ ID NO: 13 with the proviso that the amino acids at positions 105 and 106 of SEQ ID NO: 13 are substitutable such that the amino acid sequence at positions 104-106 of SEQ ID NO: 13 is NDT, NDS, NXT, or NXS, wherein X is an amino acid selected from the group consisting of A, R, N, C, E, Q, G, H, I, L, K, M, F, P, S, T, W, Y, and V.

In still another aspect, the fusion polypeptide of the present invention comprises: (a) an Ig-like domain of a first VEGF receptor that comprises an amino acid sequence that is at least 90% identical to Ig-like domain 2 of human VEGFR-1; (b) an Ig-like domain of a second VEGF receptor that comprises an amino acid sequence that is at least 90% identical to Ig-like domain 3 of human VEGFR-2; and (c) at least an Ig-like domain of a third VEGF receptor that comprises an amino acid sequence that is at least 90% identical to Ig-like domains 1 and 2 of human VEGFR-3; wherein the human VEGFR-3 has an amino acid sequence listed in SEQ ID NO: 13 with the proviso that the amino acids at positions 105 and 106 of SEQ ID NO: 13 are substitutable such that the amino acid sequence at positions 104-106 of SEQ ID NO: 13 is NDT, NDS, NXT, or NXS, wherein X is an amino acid selected from the group consisting of A, R, N, C, E, Q, G, H, I, L, K, M, F, P, S, T, W, Y, and V.

In still another aspect, the fusion polypeptide of the present invention comprises: (a) an Ig-like domain of a first VEGF receptor that comprises an amino acid sequence that is at least 95% identical to Ig-like domain 2 of human VEGFR-1; (b) an Ig-like domain of a second VEGF receptor that comprises an amino acid sequence that is at least 95% identical to Ig-like domain 3 of human VEGFR-2; and (c) at least an Ig-like domain of a third VEGF receptor that comprises an amino acid sequence that is at least 95% identical to Ig-like domains 1 and 2 of human VEGFR-3; wherein the human VEGFR-3 has an amino acid sequence listed in SEQ ID NO: 13 with the proviso that the amino acids at positions 105 and 106 of SEQ ID NO: 13 are substitutable such that the amino acid sequence at positions 104-106 of SEQ ID NO: 13 is NDT, NDS, NXT, or NXS, wherein X is an amino acid selected from the group consisting of A, R, N, C, E, Q, G, H, I, L, K, M, F, P, S, T, W, Y, and V.

In still another aspect, a fusion or chimeric polypeptide of the present invention comprises: (a) Ig-like domain 2, or substantially Ig-like domain 2, of human VEGFR-1 (or fit-1); (b) Ig-like domain 3, or substantially Ig-like domain 3, of human VEGFR-2 (or KDR); and (c) Ig-like domains 1 and 2, or substantially Ig-like domains 1 and 2, of human VEGFR-3 (or flt-4); or Ig-like domains 1, 2, and 3, or substantially Ig-like domains 1, 2, and 3, of human VEGFR-3.

In still another aspect, a fusion or chimeric polypeptide of the present invention comprises: (a) Ig-like domain 2, or substantially Ig-like domain 2, of human VEGFR-1; (b) Ig-like domain 3, or substantially Ig-like domain 3, of human VEGFR-2; and (c) Ig-like domains 1 and 2, or substantially Ig-like domains 1 and 2, of human VEGFR-3; or Ig-like domains 1, 2, and 3, or substantially Ig-like domains 1, 2, and 3, of human VEGFR-3; wherein the human VEGFR-3 has the amino acid sequence listed in SEQ ID NO: 13 with the proviso that the amino acids at positions 105 and 106 of SEQ ID NO: 13 are substitutable such that the amino acid sequence at positions 104-106 of SEQ ID NO: 13 is NDT, NDS, NXT, or NXS, wherein X is an amino acid selected from the group consisting of A, R, N, C, E, Q, G, H, I, L, K, M, F, P, S, T, W, Y, and V.

In still another aspect, a fusion or chimeric polypeptide of the present invention comprises: (a) Ig-like domain 2, or substantially Ig-like domain 2, of human VEGFR-1; (b) Ig-like domain 3, or substantially Ig-like domain 3, of human VEGFR-2; and (c) Ig-like domains 1 and 2, or substantially Ig-like domains 1 and 2, of human VEGFR-3; or Ig-like domains 1, 2, and 3, or substantially Ig-like domains 1, 2, and 3, of human VEGFR-3; wherein the human VEGFR-3 has the amino acid sequence listed in SEQ ID NO: 13 with the proviso that the amino acid sequence at positions 104-106 of SEQ ID NO: 13 is unsubstituted and remains NDT.

In still another aspect, a fusion or chimeric polypeptide of the present invention comprises: (a) Ig-like domain 2, or substantially Ig-like domain 2, of human VEGFR-1; (b) Ig-like domain 3, or substantially Ig-like domain 3, of human VEGFR-2; and (c) Ig-like domains 1 and 2, or substantially Ig-like domains 1 and 2, of human VEGFR-3; or Ig-like domains 1, 2, and 3, or substantially Ig-like domains 1, 2, and 3, of human VEGFR-3; wherein the Ig-like domain 1 of the human VEGFR-3 has an amino acid sequence listed in SEQ ID NO: 14; wherein the amino acids at positions 81 and 82 of SEQ ID NO: 14 are substitutable such that the amino acid sequence at positions 80-82 of SEQ ID NO: 14 is NDT, NDS, NXT, or NXS, wherein X is an amino acid selected from the group consisting of A, R, N, C, E, Q, G, H, I, L, K, M, F, P, S, T, W, Y, and V.

In still another aspect, a fusion or chimeric polypeptide of the present invention comprises: (a) Ig-like domain 2, or substantially Ig-like domain 2, of human VEGFR-1; (b) Ig-like domain 3, or substantially Ig-like domain 3, of human VEGFR-2; and (c) Ig-like domains 1 and 2, or substantially Ig-like domains 1 and 2, of human VEGFR-3; or Ig-like domains 1 and 2, or substantially Ig-like domains 1 and 2, of human VEGFR-3; wherein the Ig-like domain 1 of the human VEGFR-3 has an amino acid sequence listed in SEQ ID NO: 14; wherein the amino acids at positions 81 and 82 of SEQ ID NO: 14 are substitutable such that the amino acid sequence at positions 80-82 of SEQ ID NO: 14 is NDT, NDS, NXT, or NXS, wherein X is an amino acid selected from the group consisting of A, R, N, C, E, Q, G, H, I, L, K, M, F, P, S, T, W, Y, and V.

In still another aspect, a fusion or chimeric polypeptide of the present invention comprises: (a) Ig-like domain 2, or substantially Ig-like domain 2, of human VEGFR-1; (b) Ig-like domain 3, or substantially Ig-like domain 3, of human VEGFR-2; and (c) Ig-like domains 1 and 2, or substantially Ig-like domains 1 and 2, of human VEGFR-3; or Ig-like domains 1 and 2, or substantially Ig-like domains 1 and 2, of human VEGFR-3; wherein the Ig-like domain 1 of the human VEGFR-3 has an amino acid sequence listed in SEQ ID NO: 14; wherein the amino acid sequence at positions 80-82 of SEQ ID NO: 14 is unsubstituted and remains NDT.

In still another aspect, a fusion or chimeric polypeptide of the present invention comprises: (a) Ig-like domain 2, or substantially Ig-like domain 2, of human VEGFR-1; (b) Ig-like domain 3, or substantially Ig-like domain 3, of human VEGFR-2; and (c) Ig-like domains 1 and 2, or substantially Ig-like domains 1 and 2, of human VEGFR-3; or Ig-like domains 1, 2, and 3, or substantially Ig-like domains 1, 2, and 3, of human VEGFR-3; wherein the Ig-like domain 1 of human VEGFR-3 included in the fusion or chimeric polypeptide has an amino acid sequence listed in SEQ ID NO: 14.

In still another aspect, a fusion or chimeric polypeptide of the present invention comprises: (a) Ig-like domain 2, or substantially Ig-like domain 2, of human VEGFR-1; (b) Ig-like domain 3, or substantially Ig-like domain 3, of human VEGFR-2; and (c) Ig-like domains 1 and 2, or substantially Ig-like domains 1 and 2, of human VEGFR-3; or Ig-like domains 1, 2, and 3, or substantially Ig-like domains 1, 2, and 3, of human VEGFR-3; wherein the Ig-like domain 1 of human VEGFR-3 included in the fusion or chimeric polypeptide has an amino acid sequence listed in SEQ ID NO: 14, and the amino acid sequence at positions is unsubstituted and remains NDT.

In yet another aspect, the fusion or chimeric polypeptide of the present invention comprises: (a) Ig-like domain 2, or substantially Ig-like domain 2, of human VEGFR-1; (b) Ig-like domain 3, or substantially Ig-like domain 3, of human VEGFR-2; and (c) Ig-like domains 1 and 2, or substantially Ig-like domains 1 and 2, of human VEGFR-3; linked to a multimerizing component; wherein the human VEGFR-3 has the amino acid sequence listed in SEQ ID NO: 13 with the proviso that the amino acids at positions 105 and 106 of SEQ ID NO: 13 are substitutable such that the amino acid sequence at positions 104-106 of SEQ ID NO: 13 is NDT, NDS, NXT, or NXS, wherein X is an amino acid selected from the group consisting of A, R, N, C, E, Q, G, H, I, L, K, M, F, P, S, T, W, Y, and V.

In one embodiment, said multimerizing component comprises substantially a portion of an Fc domain of human IgG1.

In another embodiment, said multimerizing component comprises substantially a portion of an Fc domain of the heavy chain of human IgG1. In one embodiment, such portion of an Fc domain of the heavy chain of human IgG1 comprises the last two domains at the carboxy terminus of the IgG1 heavy chain.

In some embodiments, two or more binding units act together to bind a single ligand molecule (wherein the ligand may comprise a monomer or dimer). In some other embodiments, the binding units act independently, i.e., each binding unit binds a separate ligand molecule.

In one aspect, the fusion or chimeric protein comprises (a) Ig-like domain 2, or substantially Ig-like domain 2, of human VEGFR-1 operatively linked to (b) Ig-like domain 3, or substantially Ig-like domain 3, of human VEGFR-2, which is operatively linked to (c)(1) Ig-like domains 1 and 2, or substantially Ig-like domains 1 and 2, or (c)(2) Ig-like domains 1, 2 and 3, or substantially Ig-like domains 1, 2, and 3, of human VEGFR-3, which is operatively linked to a portion of the Fc domain of IgG1.

In one embodiment, a polypeptide linker is inserted between the last binding unit and said portion of the Fc domain of IgG1.

In one aspect, a fusion or chimeric protein comprises (a) Ig-like domain 2, or substantially Ig-like domain 2, of human VEGFR-1 operatively linked to (b) Ig-like domain 3, or substantially Ig-like domain 3, of human VEGFR-2, which is operatively linked to (c) (1) Ig-like domains 1 and 2, or substantially Ig-like domains 1 and 2, or (c)(2) Ig-like domains 1, 2, and 3, or substantially Ig-like domains 1, 2, and 3, of human VEGFR-3, which is operatively linked to a portion of the Fc domain of IgG1; wherein the Ig-like domain 2 of human VEGFR-1 (“VEGFR-1-D2”) has an amino acid sequence listed in SEQ ID NO: 2; the Ig-like domain 3 of human VEGFR-2 (“VEGFR-2-D3”) has an amino acid sequence listed in SEQ ID NO: 4; the Ig-like domains 1 and 2 of human VEGFR-3 (“VEGFR-3-D1D2”) have an amino acid sequence listed in SEQ ID NO: 6; the Ig-like domains 1, 2, and 3 of human VEGFR-3 (“VEGFR-3-D1D2D3”) have an amino acid sequence listed in SEQ ID NO: 15; and the portion of the Fc domain of IgG1 (“IgG1Fc”) has an amino acid sequence listed in SEQ ID NO: 8.

In another aspect, a fusion or chimeric polypeptide or protein of the present invention further comprises a polypeptide linker that is inserted between the carboxy terminus of VEGFR-3-D1D2 and the amino terminus of IgG1Fc; wherein said linker has an amino acid sequence listed in SEQ ID NO: 10.

In still another aspect, a fusion or chimeric polypeptide or protein of the present invention further can comprise a polypeptide leader sequence that precedes the amino terminus of VEGFR-1-D2.

In yet another aspect, a fusion or chimeric polypeptide or protein of the present invention comprises amino acid sequences that are at least 90% identical to each of SEQ ID NO: 2, SEQ ID NO: 4, SEQ ID NO: 6, SEQ ID NO: 15, SEQ ID NO: 8, and SEQ ID NO: 10; wherein the amino acids at positions 81 and 82 of SEQ ID NO: 6 and SEQ ID NO: 15 are substitutable such that the amino acid sequence at positions 80-82 of SEQ ID NO: 6 or SEQ ID NO: 15 is NDT, NDS, NXT, or NXS, wherein X is an amino acid selected from the group consisting of A, R, N, C, E, Q, G, H, I, L, K, M, F, P, S, T, W, Y, and V.

In still another aspect, a fusion or chimeric polypeptide or protein of the present invention comprises amino acid sequences that are at least 95% identical to SEQ ID NO: 2, SEQ ID NO: 4, SEQ ID NO: 6, SEQ ID NO: 15, SEQ ID NO: 8, and SEQ ID NO: 10; wherein the amino acids at positions 81 and 82 of SEQ ID NO: 6 and SEQ ID NO: 15 are substitutable such that the amino acid sequence at positions 80-82 of SEQ ID NO: 6 or SEQ ID NO: 15 is NDT, NDS, NXT, or NXS, wherein X is an amino acid selected from the group consisting of A, R, N, C, E, Q, G, H, I, L, K, M, F, P, S, T, W, Y, and V.

In still another aspect, a fusion or chimeric polypeptide or protein of the present invention comprises the amino acid sequence listed in SEQ ID NO: 12 or SEQ ID NO: 16. The fusion proteins having SEQ ID NO: 12 and SEQ ID NO: 16 were prepared for testing and are referred to herein as “EB-101” and “EB-101BIb,” respectively.

In still another aspect, a fusion or chimeric polypeptide or protein of the present invention comprises an amino acid sequence that is at least 90% identical to SEQ ID NO: 12 or SEQ ID NO: 16; wherein the amino acids at positions 286 and 287 of SEQ ID NO: 12 or SEQ ID NO: 16 are substitutable such that the amino acid sequence at positions 285-287 of SEQ ID NO: 12 or SEQ ID NO: 16 is NDT, NDS, NXT, or NXS, wherein X is an amino acid selected from the group consisting of A, R, N, C, E, Q, G, H, I, L, K, M, F, P, S, T, W, Y, and V.

In still another aspect, a fusion or chimeric polypeptide or protein of the present invention comprises an amino acid sequence that is at least 90% identical to SEQ ID NO: 12 or SEQ ID NO: 16; wherein the amino acids at positions 285-287 of SEQ ID NO: 12 or SEQ ID NO: 16 are unsubstituted.

In still another aspect, a fusion or chimeric polypeptide or protein of the present invention comprises an amino acid sequence that is at least 95% identical to SEQ ID NO: 12 or SEQ ID NO: 16; wherein the amino acids at positions 286 and 287 of SEQ ID NO: 12 or SEQ ID NO: 16 are substitutable such that the amino acid sequence at positions 285-287 of SEQ ID NO: 12 or SEQ ID NO: 16 is NDT, NDS, NXT, or NXS, wherein X is an amino acid selected from the group consisting of A, R, N, C, E, Q, G, H, I, L, K, M, F, P, S, T, W, Y, and V.

In still another aspect, a fusion or chimeric polypeptide or protein of the present invention comprises an amino acid sequence that is at least 95% identical to SEQ ID NO: 12 or SEQ ID NO: 16; wherein the amino acids at positions 285-287 of SEQ ID NO: 12 or SEQ ID NO: 16 are unsubstituted.

In still another aspect, one or more amino acid substitutions can be made in anyone of the above-described amino acid sequences. Preferably, such substitution is a conserved substitution, wherein an amino acid in one of the following groups is substituted with another in the same group: (1) A, S, T; (2) D, E; (3) N, Q; (4)R, K; (5) I, L, M, V; and (6) F, Y, W; and such substitution is selected so as to preserve substantially the binding activity of the fusion polypeptide. In one embodiment, the fusion polypeptide of the present invention having a conserved substitution has a K_{d} value less than about 120% of that before such substitution. Preferably, the K_{d} value is less than about 110% of that before such substitution. More preferably, the K_{d} value is less than about 105% of that before such substitution.

Most conserved substitutions are not expected to produce radical changes in the characteristics of the Ig-like domain or domains of the fusion polypeptide. However, when it is difficult to predict the exact effect of the substitution in advance of doing so, one skilled in the art will appreciate that the effect will be evaluated by routine screening assays. For example, an Ig-like domain variant typically is made by site-specific mutagenesis of the nucleic acid encoding the intact fusion polypeptide, expression of the variant nucleic acid in recombinant cell culture, purification of the variant fusion polypeptide from the cell culture and detecting the ability of the variant fusion polypeptide to specifically bind to a VEGF ligand. An exemplary binding assay which can be employed to determine if a particular substitution or substitutions in an Ig-like domain or domains affect the capability of the fusion polypeptide to bind to and inhibit the activity of a VEGF family member is described in the article by Park et al., J. Biol. Chem. 269:25646-25654 (1994).

The VEGFR-1-D2 binding unit of the fusion protein is capable of binding free VEGF-A, VEGF-B, and PGF with high affinity (Davis-Smyth et al., EMBO J., 15(18):4919 (1996)). The VEGFR-2-D3 binding unit of the fusion protein is capable of binding free VEGF-A, VEGF-C, and VEGF-D with high affinity (Stuttfeld et al., 61(9):915 (2009)). The VEGFR-3-D1D2 or VEGFR-3-D1D2D3 (Ig-like domains 1, 2, and 3 of VEGFR-3) binding unit of the fusion protein is capable of binding free VEGF-C and VEGF-D with high affinity. Thus, a fusion protein of the present invention is capable of substantially inhibiting the angiogenic activity of these VEGF family members on endothelial cells at the site of the disease.

In still another aspect, the present invention provides an isolated nucleic acid molecule encoding said fusion or chimeric polypeptide.

In yet another aspect, the present invention provides an isolated nucleic acid molecule encoding said fusion or chimeric polypeptide; wherein said isolated nucleic acid molecule comprises: (a) a nucleic acid sequence encoding Ig-like domain 2, or substantially Ig-like domain 2, of human VEGFR-1 (or fit-1); (b) a nucleic acid sequence encoding Ig-like domain 3, or substantially Ig-like domain 3, of human VEGFR-2 (or KDR); and (c) a nucleic acid sequence encoding Ig-like domain 1 and 2, or substantially Ig-like domains 1 and 2, of human VEGFR-3 (or fit-4).

In still another aspect, the present invention provides an isolated nucleic acid molecule encoding said fusion or chimeric polypeptide; wherein said isolated nucleic acid molecule comprises: (a) a nucleic acid sequence encoding Ig-like domain 2, or substantially Ig-like domain 2, of human VEGFR-1 (or fit-1); (b) a nucleic acid sequence encoding Ig-like domain 3, or substantially Ig-like domain 3, of human VEGFR-2 (or KDR); (c)(1) a nucleic acid sequence encoding Ig-like domains 1 and 2, or substantially Ig-like domains 1 and 2, or (c)(2) a nucleic acid sequence encoding Ig-like domains 1, 2, and 3, or substantially Ig-like domains 1, 2, and 3, of human VEGFR-3 (or fit-4); and (d) a nucleic acid sequence encoding a portion of the Fc domain of IgG1.

In still another aspect, the present invention provides an isolated nucleic acid molecule encoding said fusion or chimeric polypeptide; wherein said isolated nucleic acid molecule comprises: (a) a nucleic acid sequence encoding Ig-like domain 2, or substantially Ig-like domain 2, of human VEGFR-1 (or fit-1) and having a sequence listed in SEQ ID NO: 1; (b) a nucleic acid sequence encoding Ig-like domain 3, or substantially Ig-like domain 3, of human VEGFR-2 (or KDR) and having a sequence listed in SEQ ID NO: 3; (c) a nucleic acid sequence encoding Ig-like domain 1 and 2, or substantially Ig-like domains 1 and 2, of human VEGFR-3 (or flt-4) and having a sequence listed in SEQ ID NO: 5; and (d) a nucleic acid sequence encoding a portion of the Fc domain of IgG1 and having a sequence listed in SEQ ID NO: 7.

In still another aspect, the present invention provides an isolated nucleic acid molecule encoding said fusion or chimeric polypeptide; wherein said isolated nucleic acid molecule comprises: (a) a nucleic acid sequence encoding an amino acid sequence listed in SEQ ID NO: 2; (b) a nucleic acid sequence encoding an amino acid sequence listed in SEQ ID NO: 4; (c) a nucleic acid sequence encoding an amino acid sequence listed in SEQ ID NO: 6 or SEQ ID NO: 15; and (d) a nucleic acid sequence encoding an amino acid sequence of a portion of the Fc domain of IgG1 listed in SEQ ID NO: 8.

In yet another aspect, said isolated nucleic acid molecule further comprises a linking nucleic acid sequence encoding a polypeptide linker; wherein said linking nucleic acid has a sequence listed in SEQ ID NO: 9 and is inserted between SEQ ID NO: 5 and SEQ ID NO: 7.

In yet another aspect, said isolated nucleic acid further can comprise a leading nucleic acid sequence encoding a polypeptide leader sequence. When said leading nucleic acid sequence is present in said isolated nucleic acid, it precedes SEQ ID NO: 1.

In still another aspect, the present invention provides an isolated nucleic acid molecule encoding said fusion or chimeric polypeptide; wherein said isolated nucleic acid molecule comprises a nucleic acid sequence listed in SEQ ID NO: 11.

In still another aspect, the present invention provides an isolated nucleic acid molecule encoding a fusion or chimeric polypeptide or protein; wherein said fusion or chimeric polypeptide or protein has SEQ ID NO: 12 or SEQ ID NO: 16.

In another aspect, the present invention provides an isolated nucleic acid molecule encoding said fusion or chimeric polypeptide; wherein said isolated nucleic acid molecule comprises a nucleic acid sequence that, as a result of the degeneracy of the genetic code, differs in one or more codons from a nucleic acid sequence selected from the group consisting of SEQ ID NO: 1, SEQ ID NO: 3, SEQ ID NO: 5, SEQ ID NO: 7, SEQ ID NO: 9, and SEQ ID NO: 11. Such different nucleic acid sequence is within the scope of the present invention.

In still another aspect, the present invention provides a vector that comprises any of said nucleic acid molecules, including an expression vector comprising any of said nucleic molecules operatively linked to an expression control sequence. Embodiments of the vector comprise a nucleic acid sequence selected from the group consisting of SEQ ID NO: 1, SEQ ID NO: 3, SEQ ID NO: 5, SEQ ID NO: 7, SEQ ID NO: 9, and SEQ ID NO: 11. In particular, a vector of the present invention comprises a nucleic acid sequence selected from the group consisting of SEQ ID NO: 1, SEQ ID NO: 3, SEQ ID NO: 5, and SEQ ID NO: 11.

In still another aspect, the present invention provides a host-vector system for the production of said fusion or chimeric polypeptide that comprises the expression vector in a suitable host cell.

In one aspect, the present invention provides for the construction of a nucleic acid molecule encoding a fusion polypeptide disclosed herein, which nucleic acid is inserted into a vector that is able to express the fusion polypeptide when introduced into an appropriate host cell. Appropriate host cells include, but are not limited to, bacterial cells, yeast cells, insect cells, and mammalian cells. Any of the methods known to one skilled in the art for the insertion of DNA fragments into a vector may be used to construct expression vectors encoding the chimeric polypeptide molecules under control of transcriptional/translational control signals. These methods may include in vitro recombinant DNA and synthetic techniques and in vivo recombinations (genetic recombination) (See Sambrook, et al., Molecular Cloning, A Laboratory Manual, Cold Spring Harbor Laboratory; Current Protocols in Molecular Biology, Eds. Ausubel, et al., Greene Publ. Assoc., Wiley-Interscience, NY).

Expression of a nucleic acid molecule encoding a fusion polypeptide of the present invention may be regulated by a second nucleic acid sequence (a promoter) so that the fusion polypeptide is expressed in a host transformed with the nucleic acid molecule. For example, expression of the fusion polypeptide described herein may be controlled by any promoter/enhancer element known in the art.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli is typically transformed using pBR322, a plasmid derived from an E. coli species (see; e.g., Bolivar et al., Gene, 2:95 (1977)). The plasmid pBR322 contains genes for ampicillin and tetracycline resistance and thus provides easy means for identifying transformed cells. The pBR322 plasmid, or other microbial plasmid or phage, must also contain, or be modified to contain, promoters that can be used by the microbial organism for expression of proteins.

Those promoters most commonly used in recombinant DNA construction include the β-lactamase (penicillinase) and lactose promoter systems or a tryptophan (trp) promoter system (Goeddel et al., Nucleic Acids Res., 8:4057 (1980)). While these are the most commonly used, other microbial promoters have been discovered and utilized. For example, the tac promoter is a synthetically produced DNA promoter produced from the combination of promoters from the trp and lac operons (de Boer et al., PNAS, (1983-01-80 (1):21-25 (1983)). It is commonly used for protein production in Escherichia coli. (Amann et al., Gene, 25:167 (1983)). Any of these promoters may be used in connection with a method of producing a fusion polypeptide of the present invention.

In addition to prokaryotes, eukaryotic microbes, such as yeast cultures, may also be used. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al., Nature, 282:39 (1979)) is commonly used. Other plasmids are disclosed in U.S. Pat. No. 4,615,974; Struhl et al., PNAS, 76(3):1035 (1979). The plasmid YRp7 contains the trp1 gene that provides a selection marker for a mutant strain of yeast lacking the ability to grow without tryptophan, for example, ATCC No. 44,076 or RH218 (Jones, Genetics, 85:23 (1977)). The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzeman et al., J. Biol. Chem., 255:2073 (1980)) or other glycolytic enzymes, such as glyceraldehyde-3-phosphate dehydrogenase, hexokinase, pyruvate decarboxylas, and glucokinase (Romanos et al., Yeast, 8:423 (1992); Weinhandl et al., Microb. Cell factories, 13:5 (2014)). In constructing suitable expression plasmids, the termination sequences associated with these genes are also ligated into the expression vector 3′ of the sequence desired to be expressed to provide polyadenylation of the mRNA and termination. Other promoters, which have the additional advantage of transcription controlled by growth conditions, such as the promoter region for alcohol dehydrogenase 2, and enzymes responsible for maltose and galactose utilization (Romanos et al., Weinhandl et al., supra). Any plasmid vector containing yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, much interest has been in vertebrate cells, and propagation of vertebrate cells in culture (tissue culture) has become a routine procedure in recent years (Tissue Culture, Academic Press, Kruse and Patterson, editors (1973)). Examples of such useful host cell lines are VERO and HeLa cells, Chinese hamster ovary (CHO) cell lines, and W138, BHK, COS-7, 293, and MDCK cell lines. Expression vectors for such cells ordinarily include (if necessary) an origin of replication, a promoter located in front of the gene to be expressed, along with any necessary ribosome binding sites, RNA splice sites, polyadenylation sites, and transcriptional terminator sequences.

For use in mammalian cells, the control functions on the expression vectors are often provided by viral material. For example, commonly used promoters are derived from polyoma, Adenovirus 2, and most frequently Simian Virus 40 (SV40). The early and late promoters of SV40 virus are particularly useful because both are obtained easily from the virus as a fragment that also contains the SV40 viral origin of replication (Fiers et al., Nature, 273:113 (1978)). Smaller or larger SV40 fragments may also be used, provided there is included the approximately 250-bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

Thus, according to the invention, expression vectors capable of being replicated in a bacterial, a yeast cell, an insect cell, or a mammalian cell host, comprising a fusion polypeptide-encoding nucleic acid as described herein, are used to transfect the host and thereby direct expression of such nucleic acids to produce the fusion polypeptide, which may then be recovered in a biologically active form. As used herein, a biologically active form includes a form capable of binding to at least a VEGF family member.

In some embodiments, the host cell can be E. coli, a COS cell, or a Chinese hamster ovary (“CHO”) cell. In some other embodiments, the host cell can be HEK293 cell.

### Vector Construction

Construction of suitable vectors containing the desired coding and control sequences employ standard ligation techniques. Isolated plasmids or DNA fragments are cleaved, tailored, and ligated in the form desired to form the plasmids required. The methods employed are not dependent on the DNA source or intended host. Cleavage is performed by treating with restriction enzyme (or enzymes) in suitable buffer.

A nucleic acid sequence substantially encoding one or more Ig-like domains of VEGFR-1, VEGFR-2, or VEGR-3 can be produced according to the method disclosed in U.S. Pat. No. 6,897,294.

Nucleic acid sequences substantially encoding the Ig-like domain 2 of VEGFR-1, Ig-like domain 3 of VEGFR-2, and Ig-like domains 1 and 2 of VEGR-3 are ligated in tandem in the desired order. This construct is then ligated to the N-terminus of the nucleic acid sequence encoding the desired Fc portion of IgG1. The entire nucleic acid sequence is then positioned in a vector which contains a promoter in the reading frame with the gene and compatible with the proposed host cell. A number of plasmids, such as those described in U.S. Pat. Nos. 4,456,748; 5,460,811; 5,888,808; and 6,333,147, may be used in a production of a fusion polypeptide of the present invention.

In one embodiment, a fusion polypeptide of the present invention is produced according to the method described in U.S. Pat. No. 7,070,959. The entire nucleic acid sequence of the fusion polypeptide (“VEGFR-1-D2-VEGFR-2-D3-VEGFR-3-D1D2-Fc” or “VEGFR-1-D2-VEGFR-2-D3-VEGFR-3-D1D2D3-Fc) is inserted into the expression vector pEE14.1 (Lonza Biologics) having the CMV promoter. CHO K1 cells are transfected with the pEE14.1/VEGFR-1-D2-VEGFR-2-D3-VEGFR-3-D1D2-Fc or pEE14.1/VEGFR-1-D2-VEGFR-2-D3-VEGFR-3-D1D2D3-Fc DNA construct and then grown. The fusion polypeptide obtained from the CHO or HEK293 cells is purified and characterized by binding assay, as described in U.S. Pat. No. 7,070,959.

In one embodiment, a fusion polypeptide of the present invention can bind to VEGF family members with K_{d}≤10⁻⁹ M. In another embodiment, a fusion polypeptide of the present invention can bind to VEGF family members with K_{d}≤5×10⁻¹⁰ M. In still another embodiment, a fusion polypeptide of the present invention can bind to VEGF family members with K_{d}≤10⁻¹⁰ M.

In one aspect, the present invention provides compounds, compositions, and methods for treating or controlling a disease, condition, or disorder having etiology in aberrant angiogenesis.

In another aspect, the present invention provides a method for treating or controlling at least an ocular disease, condition, or disorder, in a subject, which has etiology in aberrant angiogenesis. The method comprises administering to a subject in need of such treating or controlling a composition comprising a fusion polypeptide that comprises an amino acid sequence selected from the group consisting of: SEQ ID NO: 2; SEQ ID NO: 4; SEQ ID NO: 6; SEQ ID NO: 15, SEQ ID NO: 12, and SEQ ID NO: 16.

In still another aspect, the method comprises administering to a subject a composition comprising a fusion polypeptide that comprises an amino acid sequence that is at least 90% identical to the amino acid sequence listed in SEQ ID NO: 12 or SEQ ID NO: 16 with the proviso that the amino acids at positions 286 and 287 of SEQ ID NO: 12 or SEQ ID NO: 16 are substitutable such that the amino acid sequence at positions 285-287 of SEQ ID NO: 12 or SEQ ID NO: 16 is NDT, NDS, NXT, or NXS, wherein X is an amino acid selected from the group consisting of A, R, N, C, E, Q, G, H, I, L, K, M, F, P, S, T, W, Y, and V.

In still another aspect, the method comprises administering to a subject a composition comprising a fusion polypeptide that comprises an amino acid sequence that is at least 90% identical to the amino acid sequence listed in SEQ ID NO: 12 or SEQ ID NO: 16 with the proviso that the amino acid sequence at positions 285-287 of SEQ ID NO: 12 or SEQ ID NO: 16 is unsubstituted and remains NDT.

In still another aspect, the method comprises administering to a subject a composition comprising a fusion polypeptide that comprises an amino acid sequence that is at least 95% identical to the amino acid sequence listed in SEQ ID NO: 12 or SEQ ID NO: 16 with the proviso that the amino acids at positions 286 and 287 of SEQ ID NO: 12 or SEQ ID NO: 16 are substitutable such that the amino acid sequence at positions 285-287 of SEQ ID NO: 12 or SEQ ID NO: 16 is NDT, NDS, NXT, or NXS, wherein X is an amino acid selected from the group consisting of A, R, N, C, E, Q, G, H, I, L, K, M, F, P, S, T, W, Y, and V.

In still another aspect, the method comprises administering to a subject a composition comprising a fusion polypeptide that comprises an amino acid sequence that is at least 95% identical to the amino acid sequence listed in SEQ ID NO: 12 or SEQ ID NO: 16 with the proviso that the amino acid sequence at positions 285-287 of SEQ ID NO: 12 or SEQ ID NO: 16 is unsubstituted and remains NDT.

In still another aspect, said ocular disease, condition, or disorder is selected from the group consisting of: choroidal neovascularization (including myopic choroidal neovascularization), polypoidal choroidal vasculopathy, retinal neovascularization, vascular leak, retinal edema, diabetic macular edema, macular edema caused by a condition selected from the group consisting of retinal vein occlusion and non-infectious posterior uveitis, diabetic retinopathy, corneal neovascularization, corneal inflammation, myopic neovascularization, neovascular glaucoma, and neovascular age-related macular degeneration (wet age-related macular degeneration resulting from retinal neovascularization).

In another aspect, the present invention provides a use of a fusion polypeptide for a preparation or manufacture of a medicament for treating or controlling at least an ocular disease, condition, or disorder, in a subject, which has etiology in aberrant angiogenesis; wherein the fusion polypeptide comprises an amino acid sequence selected from the group consisting of: SEQ ID NO: 2; SEQ ID NO: 4; SEQ ID NO: 6; SEQ ID NO: 15, SEQ ID NO: 12, and SEQ ID NO: 16.

In still another aspect, the present invention provides a use of a fusion polypeptide for the preparation or manufacture of a medicament for treating or controlling at least an ocular disease, condition, or disorder, in a subject, which has etiology in aberrant angiogenesis; wherein the fusion polypeptide comprises an amino acid sequence that is at least 90% identical to the amino acid sequence listed in SEQ ID NO: 12 or SEQ ID NO: 16 with the proviso that the amino acids at positions 286 and 287 of SEQ ID NO: 12 or SEQ ID NO: 16 are substitutable such that the amino acid sequence at positions 285-287 of SEQ ID NO: 12 or SEQ ID NO: 16 is NDT, NDS, NXT, or NXS, wherein X is an amino acid selected from the group consisting of A, R, N, C, E, Q, G, H, I, L, K, M, F, P, S, T, W, Y, and V.

In still another aspect, the fusion polypeptide for said use comprises an amino acid sequence that is at least 90% identical to the amino acid sequence listed in SEQ ID NO: 12 or SEQ ID NO: 16 with the proviso that the amino acid sequence at positions 285-287 of SEQ ID NO: 12 or SEQ ID NO: 16 is unsubstituted and remains NDT.

In still another aspect, the present invention provides a use of a fusion polypeptide for the preparation or manufacture of a medicament for treating or controlling at least an ocular disease, condition, or disorder, in a subject, which has etiology in aberrant angiogenesis; wherein the fusion polypeptide comprises an amino acid sequence that is at least 95% identical to the amino acid sequence listed in SEQ ID NO: 12 or SEQ ID NO: 16 with the proviso that the amino acids at positions 286 and 287 of SEQ ID NO: 12 or SEQ ID NO: 16 are substitutable such that the amino acid sequence at positions 285-287 of SEQ ID NO: 12 or SEQ ID NO: 16 is NDT, NDS, NXT, or NXS, wherein X is an amino acid selected from the group consisting of A, R, N, C, E, Q, G, H, I, L, K, M, F, P, S, T, W, Y, and V.

In still another aspect, the fusion polypeptide for said use comprises an amino acid sequence that is at least 95% identical to the amino acid sequence listed in SEQ ID NO: 12 or SEQ ID NO: 16 with the proviso that the amino acid sequence at positions 285-287 of SEQ ID NO: 12 or SEQ ID NO: 16 is unsubstituted and remains NDT.

In still another aspect, said ocular disease, condition, or disorder is selected from the group consisting of the diseases, conditions, or disorders herein disclosed.

In one embodiment, the subject is administered with a dose of about 25-4000 micrograms of the fusion polypeptide. In another embodiment, the subject is administered with a dose of about 50-4000, about 100-4000, about 500-4000, about 1000-4000, about 2000-4000, about 50-3000, about 50-2000, about 50-1000, or about 50-500 micrograms of the fusion polypeptide.

In still another aspect, the composition comprising the fusion polypeptide is in the form of an eye drop or an ocular injection (such as intravitreal, intracameral, peri-orbital, subtenon, subretinal, or suprachoroidal injection). Such a composition comprises an ophthalmic composition. A fusion polypeptide of the present invention may also be incorporated in a medical device that is implantable into or near a diseased tissue.

In one embodiment, for treating or controlling an anterior-disease, condition, or disorder; such as corneal neovascularization, corneal inflammation, or neovascular glaucoma, the composition comprising the fusion polypeptide may be in the form of an eye drop or intracameral or subconjunctival injection. In another embodiment, for treating or controlling a posterior-segment disease, condition, or disorder; such as choroidal neovascularization (including myopic choroidal neovascularization), neovascular age-related macular degeneration, vascular leak, retinal edema, or diabetic retinopathy, the composition comprising the fusion polypeptide may be in the form of an intravitreal injection.

In yet another aspect, an eye drop is administered to the subject at least once per day, at least once per week, or at least once per month until the disease, condition, or disorder is substantially treated or controlled.

In yet another aspect, the composition is administered to the subject for a period of at least one month, at least two months, at least three months, or at least six months.

In still another aspect, an intravitreal injection or an injection into, or near, a diseased tissue is administered to the subject according to a regimen recommended by a medical practitioner for a particular patient. For example, an injection may be administered at least once per month, at least once every two months, at least once every three months, or at least once every six months until the disease, condition, or disorder is substantially treated or controlled. In one embodiment, treatment may be administered more frequently at the beginning, and then less frequently after a period of time, as may be determined by a medical practitioner.

The concentration of a fusion polypeptide of the present invention in such an ophthalmic composition can be in the range from about 0.1 to about 200 mg/ml (or, alternatively, from about 0.25 to about 200 mg/ml, or from about 0.25 to about 160 mg/ml, or from about 0.5 to about 100 mg/ml, or from about 0.25 to about 50 mg/ml, or from about 0.5 to about 200 mg/ml, or from about 0.5 to about 160 mg/ml, or from about 0.5 to about 100 mg/ml, or from about 0.5 to about 50 mg/ml, or from about 1 to about 200 mg/ml, or from 1 to about 160 mg/ml, or from about 0.5 to about 100 mg/ml, or from about 1 to about 50 mg/ml).

In still another aspect, a method for preparing a composition of the present invention comprises combining: (a) an amount of a fusion polypeptide of the present invention; and (b) a physiologically acceptable carrier.

In one embodiment, such a physiologically acceptable carrier can be a sterile saline solution or a physiologically acceptable buffer. In another embodiment, such a carrier comprises a hydrophobic medium, such as a pharmaceutically acceptable oil. In still another embodiment, such as carrier comprises an emulsion of a hydrophobic material and water. In yet another embodiment, a fusion polypeptide of the present invention may be associated or linked with a high-molecular weight material to provide a long circulation time.

Physiologically acceptable buffers include, but are not limited to, a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10×phosphate buffer saline (“PBS”) or 5×PBS

### Solution

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis {2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis {2-hydroxyethyl}amino)-2-hydroxypropane)) having pKₐ of 7.52 at 25° C. and pH in the range of about 7-8.2; TAPSO (2-hydroxy-3 {tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In certain embodiments, a composition of the present invention is formulated in a buffer having an acidic pH, such as from about 4 to about 6.8, or alternatively, from about 5 to about 6.8. In such embodiments, the buffer capacity of the composition desirably allows the composition to come rapidly to a physiological pH after being administered into the patient.

In addition to a buffer, a composition of the present invention can comprise a material selected from the group consisting of surfactants, stabilizers, preservatives, co-solvent, humectants, emollients, chelating agents, tonicity-adjusting agents, and antioxidants.

In one aspect, any of these materials that may be used in a composition of the present invention is an ophthalmically acceptable material.

Water-soluble preservatives that may be employed include sodium bisulfite, sodium bisulfate, sodium thiosulfate, benzalkonium chloride, chlorobutanol, thimerosal, ethyl alcohol, methylparaben, polyvinyl alcohol, benzyl alcohol, and phenylethyl alcohol. These agents may be present in individual amounts of from about 0.001 to about 5% by weight (preferably, about 0.01% to about 2% by weight). Suitable water-soluble buffering agents that may be employed are sodium carbonate, sodium borate, sodium phosphate, sodium acetate, sodium bicarbonate, etc., as approved by the United States Food and Drug Administration (“US FDA”) for the desired route of administration.

Non-limiting examples of surfactants include, but are not limited to, non-ionic surfactants, for example, polysorbates (such as polysorbate 20, polysorbate 80), 4-(1,1,3,3-tetramethylbutyl)phenol/poly(oxyethylene) polymers (such as the polymer sold under the trademark Tyloxapol), poly(oxyethylene)-poly(oxypropylene) block copolymers, glycolic esters of fatty acids and the like, and mixtures thereof.

In one aspect, the pH of the composition is in the range from about 4 to about 11. Alternatively, the pH of the composition is in the range from about 6 to about 8, or from about 6.5 to about 8.

In another aspect, the composition has a pH of about 7. Alternatively, the composition has a pH in a range from about 7 to about 7.5.

In still another aspect, the composition has a pH of about 7.4.

In yet another aspect, a composition also can comprise a viscosity-modifying compound designed to facilitate the administration of the composition into the subject or to promote the bioavailability in the subject. In still another aspect, the viscosity-modifying compound may be chosen so that the composition is not readily dispersed after being administered into an environment of an eye. Such compounds may enhance the viscosity of the composition, and include, but are not limited to: monomeric polyols, such as, glycerol, propylene glycol, ethylene glycol; polymeric polyols, such as, polyethylene glycol; various polymers of the cellulose family, such as hydroxypropylmethyl cellulose (“HPMC”), carboxymethyl cellulose (“CMC”) sodium, hydroxypropyl cellulose (“HPC”); polysaccharides, such as hyaluronic acid and its salts, chondroitin sulfate and its salts, dextrans, such as, dextran 70; water soluble proteins, such as gelatin; vinyl polymers, such as, polyvinyl alcohol, polyvinylpyrrolidone, povidone; carbomers, such as carbomer 934P, carbomer 941, carbomer 940, or carbomer 974P; and acrylic acid polymers. In general, a desired viscosity can be in the range from about 1 to about 400 centipoises (“cps”) or mPa·s.

Non-limiting examples of chelating agents include ethylenediaminetetraacetic acid (“EDTA”), diethylenetriaminepentakis(methylphosphonic acid), etidronic acid, tetrasodium salt of etidronic acid (also known as “HAP”).

While the buffer itself is a “tonicity-adjusting agent” and a “pH-adjusting agent” that broadly maintains the ophthalmic solution at a particular ion concentration and pH, additional “tonicity-adjusting agents” can be added to adjust the final tonicity of the solution. Such tonicity-adjusting agents are well known to those of skill in the art and include, but are not limited to, mannitol, sorbitol, dextrose, sucrose, urea, propylene glycol, and glycerin. Also, various salts, including halide salts of a monovalent cation (e.g., NaCl or KCl) can be utilized. Typically, the tonicity of a formulation of the present invention is in the range from about 200 to 400 mOsm/kg. Alternatively, the tonicity of a formulation of the present invention is in the range from about 220 to 400 mOsm/kg, or from about 220 to 350 mOsm/kg, or from about 220 to 300 mOsm/kg, or from about 250 to 350 mOsm/kg.

Non-limiting examples of anti-oxidants include ascorbic acid (vitamin C) and its salts and esters; tocopherols (such as α-tocopherol) and tocotrienols (vitamin E), and their salts and esters (such as vitamin E TGPS (D-α-tocopheryl polyethylene glycol 1000 succinate)); glutathione; lipoic acid; uric acid; butylated hydroxyanisole (“BHA”); butylated hydroxytoluene (“BHT”); tertiary butylhydroquinone (“TBHQ”); and polyphenolic anti-oxidants (such as gallic acid, cinnanmic acid, flavonoids, and their salts, esters, and derivatives).

Non-limiting examples of stabilizers includes sucrose, mannitol, sorbitol, and trehalose.

It should be understood that the proportions of the various components or mixtures in the following examples may be adjusted for the appropriate circumstances.

In another aspect, a fusion polypeptide of the present invention and appropriate amounts of one or more desired excipients are incorporated into a formulation for topical administration or injection to a portion of the eye, such as the anterior or posterior segment, or the vitreous humor. An injectable formulation can desirably comprise a carrier that provides a sustained-release of the active ingredients, such as for a period longer than about 1 week (or longer than about 1, 2, 3, 4, 5, or 6 months).

In still another aspect, a composition comprising a fusion polypeptide of the present invention and desired excipients is lyophilized and is reconstituted with a physiologically acceptable liquid carrier substantially immediately before administration to a subject.

In one embodiment, a compound or composition of the present invention can be injected with a fine-gauge needle, such as 25-35 gauge. Typically, an amount from about 25 μl to about 100 μl of a composition comprising about 25-4000 μg of a fusion polypeptide of the present invention is administered into a patient. In one embodiment, the fusion polypeptide has an amino acid sequence that is substantially the same as that listed in SEQ ID NO:12 or SEQ ID NO: 16 with the proviso that the amino acids at positions 286 and 287 of SEQ ID NO: 12 or SEQ ID NO: 16 are substitutable such that the amino acid sequence at positions 285-287 of SEQ ID NO: 12 or SEQ ID NO: 16 is NDT, NDS, NXT, or NXS, wherein X is an amino acid selected from the group consisting of A, R, N, C, E, Q, G, H, I, L, K, M, F, P, S, T, W, Y, and V. In another embodiment, the fusion polypeptide has an amino acid sequence as listed in SEQ ID NO:12 or SEQ ID NO: 16. A concentration of such fusion polypeptide is selected from the ranges disclosed above.

In still another aspect, a fusion polypeptide of the present invention is incorporated into an ophthalmic device that comprises a biodegradable material, and the device is implanted into a posterior-segment tissue of a subject to provide a long-term (e.g., longer than about 1 week, or longer than about 1, 2, 3, 4, 5, or 6 months) treatment or control of an angiogenic disease, condition, or disorder. Such a device may be implanted by a skilled physician in the subject's ocular or periocular tissue. Non-limiting examples of ophthalmic implant systems or devices for the sustained-release of an active ingredient are disclosed in U.S. Pat. Nos. 5,378,475; 5,773,019; 5,902,598; 6,001,386; 6,051,576; and 6,726,918.

In still another aspect, a method for treating or controlling an ophthalmic angiogenic disease, condition, or disorder comprises administering a composition comprising a fusion polypeptide of the present invention to a subject in need thereof.

In still another aspect, a method for treating or controlling an ophthalmic angiogenic disease, condition, or disorder comprises administering a composition comprising a fusion polypeptide having an amino acid sequence as listed in SEQ ID NO:12 or SEQ ID NO: 16 to a subject in need thereof.

In still another aspect, a method for treating or controlling an ophthalmic angiogenic disease, condition, or disorder having an etiology in aberrant angiogenesis of in the posterior segment of an eye comprises intravitreally injecting a composition comprising a fusion polypeptide having an amino acid sequence as listed in SEQ ID NO:12 or SEQ ID NO: 16 in a subject in need thereof.

In another embodiment, such disease, condition, or disorder is selected from the group consisting of choroidal neovascularization, polypoidal choroidal vasculopathy, myopic neovascularization, retinal neovascularization including retinopathy of prematurity, vascular leak, retinal edema including diabetic macular edema and macular edema caused by other retinal conditions such as retinal vein occlusion and non-infectious posterior uveitis, diabetic retinopathy including proliferative diabetic retinopathy, corneal neovascularization, and neovascular glaucoma

In yet another aspect, a composition of the present invention is administered once a week, once a month, once a year, twice a year, four times a year, or at a suitable frequency that is determined to be appropriate for treating or controlling an anterior-segment inflammatory disease, condition, or disorder.

In still another aspect, a fusion polypeptide or protein of the present invention can also be used for the treatment or control of other non-ocular diseases or conditions that are promoted by, or have etiology in, aberrant angiogenesis, such as cancers, psoriasis, rheumatoid arthritis, and atherosclerosis. Such treatment or control may be effected by, for example, systemic administration.

In yet another aspect, the present invention provides a use of a fusion polypeptide or protein for the preparation or manufacture of a medicament for treating or controlling of non-ocular diseases or conditions that are promoted by, or have etiology in, aberrant angiogenesis, such as cancers, psoriasis, rheumatoid arthritis, and atherosclerosis.

The cancer amenable for treatment by the present invention include, but not limited to, carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome. In some embodiments, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, melanoma, ovarian cancer, mesothelioma, and multiple myeloma. In another embodiment, the cancer is colorectal cancer. The cancerous conditions amenable for treatment of the invention include metastatic cancers. The method of the present invention is particularly suitable for the treatment of vascularized tumors.

### Example 1: Composition 1

A composition of the present invention is prepared by combining 10-100 mg/mL of the fusion protein EB-101, 10 mM histidine HCl buffer, 10% (by weight) α,α-trehalose, and (by weight) polysorbate 20. In some embodiments, the composition may also include a material selected from the group consisting of an antioxidant, a chelating agent, and a preservative, and combinations thereof. The composition has a pH in the range of about 5-6 and is suitable for intraocular, including intravitreal, administration.

### Example 2: Composition 2

A composition of the present invention is prepared by combining 10-100 mg/mL of the fusion protein EB-101, 10 mM phosphate buffer, 40 mM NaCl, 5% (by weight) sucrose, and (by weight) polysorbate 20. The composition has a pH in the range of about 5.5-6.5 and is suitable for intraocular, including intravitreal, administration.

### Example 3: Transient Expression of EB-101 in HEK293 Cells

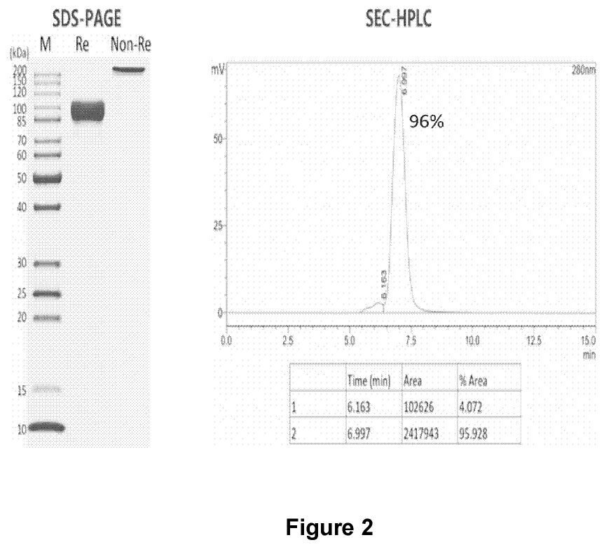

Gene encoding the amino acid sequence of EB-101 as indicated by the SEQ ID NO: 12 was synthetized and a vector for expressing the protein was constructed. The expression vector was used to transiently transfect 293 cells with chemically defined culture media. The produced protein was purified by Protein A affinity column, ultrafiltration and then subjected to 0.2 μm sterile filtration to get the bulk of high purity. Expression of EB-101 in HEK293 cells produced a protein with a MW ˜200KDa (non-reduced form at SDS-PAGE) and a purity of 96% when analyzed by size exclusion-chromatography (SEC-HPLC). See FIG. 2.

### Example 4: Transient Expression of EB-101 in CHO Cells

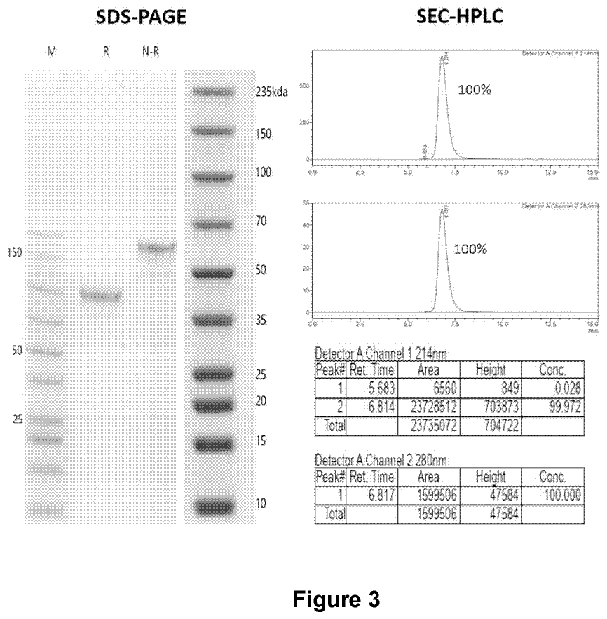

Gene encoding the amino acid sequence of EB-101 as indicated by the SEQ ID NO: 12 was synthetized and a vector for expressing the protein was constructed. The expression vector was used to transiently transfect CHO cells with chemically defined culture media. The produced protein was purified by Protein A affinity column, ultrafiltration and then subjected to sterile filtration to get the bulk of high purity. Expression of EB-101 in CHO cells produced a protein with a MW ˜200KDa (non-reduced form at SDS-PAGE) and a purity of 100% when analyzed by SEC-HPLC. See FIG. 3.

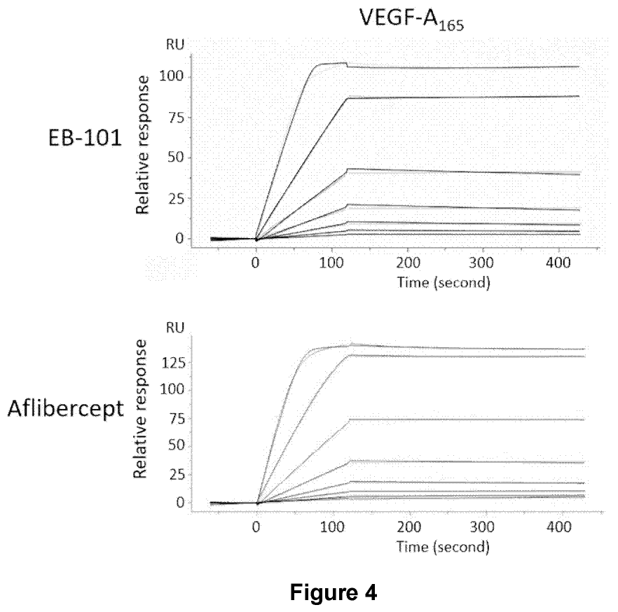

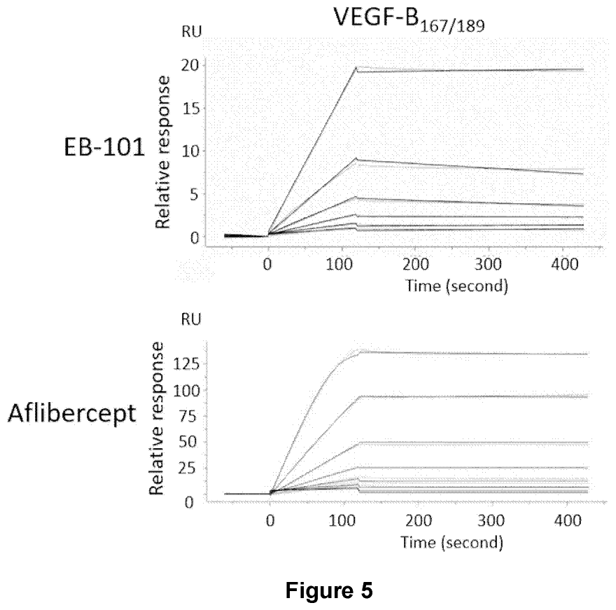

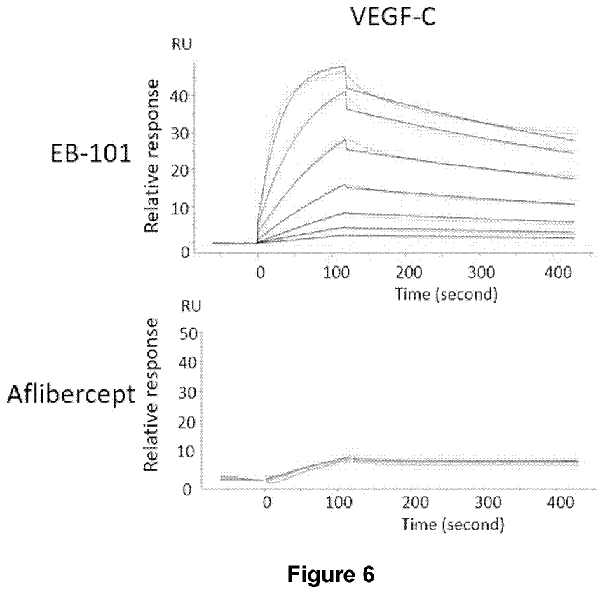

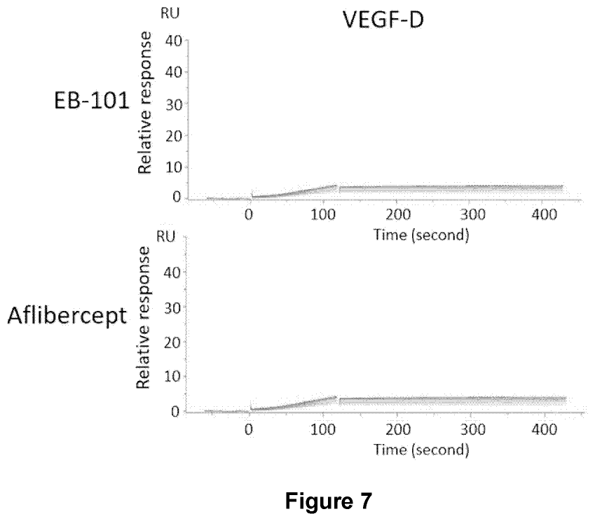

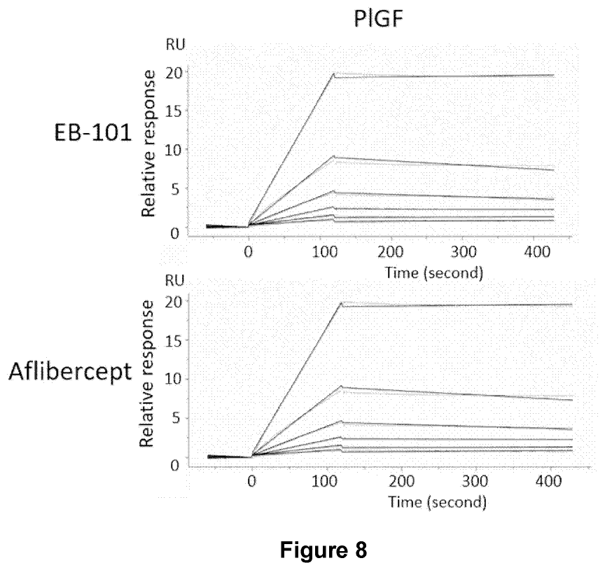

### Example 5: Comparison of the Binding Affinity of EB-101 with Aflibercept for Human Recombinant Human VEGF-A₁₆₅, VEGF-B_{167/189}, VEGF-C, VEGF-D, and PlGF Using SPR Biacore. See FIGS. 4-8

SPR Biacore assays were performed by immobilization of the anti-human IgG (Fc) antibody onto the CMS sensor chip surface and determined the level of ligand immobilization. The amount of anti-Fc antibody coupling onto the CMS sensor chip was about 7,000˜14,000 response unit (“RU”). Then, EB-101 or aflibercept were injected into the sample channel to reach a capture level of about 400 RU. Finally, different concentrations of the analyte (VEGF-A in this assay) diluted in running buffer were injected for affinity and kinetics measurements. The 1:1 binding model was used to measure the binding affinity and/or kinetics. The kinetic constants of VEGF-A₁₆₅ binding to EB-101 were as follows: equilibrium dissociation constant K_{D}=7.51×10⁻¹² M (7.51 pM), association rate constant Kₐ=5.06×10⁷ M⁻¹ s⁻¹, dissociation rate constant Kₐ=3.64×10⁻⁴ s⁻¹. The kinetic constants of VEGF-A₁₆₅ binding to aflibercept were as follows: equilibrium dissociation constant K_{D}=3.36×10⁻¹¹ M (33.6 pM), association rate constant Kₐ=1.36×10⁷ M⁻¹ s⁻¹, dissociation rate constant Kₐ=4.58×10⁻⁴ s⁻¹.

The kinetic constants of VEGF-B_{167/189} binding to EB-101 were as follows: equilibrium dissociation constant K_{D}=1.38×10⁻¹⁰ M (138 pM), association rate constant Kₐ=2.20×10⁶ M⁻¹ s⁻¹, dissociation rate constant K_{d}=3.03×10⁻⁴ s⁻¹. The kinetic constants of VEGF-B_{167/189} binding to aflibercept were as follows: equilibrium dissociation constant K_{D}=1.73×10⁻¹¹ M (17.3 pM), association rate constant Kₐ=3.27×10⁶ M⁻¹ s⁻¹, dissociation rate constant K_{d}=5.67×10⁻⁵ s⁻¹.

The kinetic constants of VEGF-C binding to EB-101 were as follows: equilibrium dissociation constant K_{D}=4.64×10⁻¹⁰ M (0.464 nM), association rate constant Kₐ=3.11×10⁶ M⁻¹ s⁻¹, dissociation rate constant K_{d}=1.44×10⁻³ s⁻¹. The binding between VEGF-C and aflibercept cannot be detected.

The binding between VEGF-D and EB-101 or aflibercept cannot be detected.

The kinetic constants of P1GF binding to EB-101 were as follows: equilibrium dissociation constant K_{D}=2.09×10⁻¹⁰ M (209 pM), association rate constant Kₐ=1.68×10⁷ M⁻¹ s⁻¹, dissociation rate constant K_{d}=3.15×10⁻³ s⁻¹. The kinetic constants of P1GF binding to aflibercept were as follows: equilibrium dissociation constant K_{D}=4.84×10⁻¹⁰ M (484 pM), association rate constant Kₐ=1.26×10⁶M⁻¹ s⁻¹, dissociation rate constant K_{d}=6.10×10⁻⁴ s⁻¹.

TABLE 1
Comparison of the binding affinities of EB-101
and aflibercept for VEGF-A₁₆₅, VEGF-B_{167/189},
VEGF-C, VEGF-D, and PlGF using SPR Biacore

| K_{D} (SPR | | VEGF- | | VEGF- | VEGF- | |
|---|---|---|---|---|---|---|
| Biacore) | VEGFA₁₆₅ | B_{167/189} | | C | D | PlGF |
| EB-101 | 7.51 pM | 0.14 | nM | 0.46 nM | no | 0.21 nM |
| | | | | | binding | |
| Aflibercept | 33.60 pM | 17.30 | pM | no | no | 0.48 nM |
| | | | | binding | binding | |

Thus, EB-101 binding to these VEGF family members compares more favorably than aflibercept.

### Example 6: Comparison of the Effect of EB-101 with Aflibercept and Bevacizumab on Inhibition of VEGF-A₁₆₅ Mediated VEGFR-2 Signaling in VEGFR-2-NFAT-RE Luciferase Reporter Cells

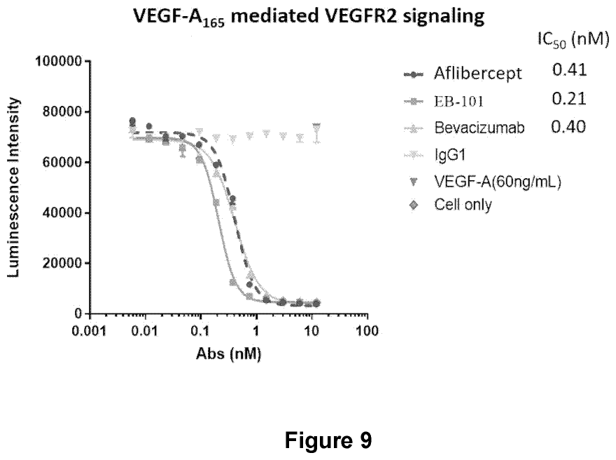

Recombinant human VEGF-A 165 were mixed with serial dilutions of the test compound, incubated at room temperature for 30 minutes, and then added into wells containing 4×10⁴ VEGFR-2 (KDR)-NFAT-RE luciferase reporter cells/well followed by an incubation at 37° C. for 6 hours. Luciferase signal was detected by a plate reader after addition of 50 μl of Bright-Lite. The effect of EB-101 on inhibiting VEGF-A₁₆₅ mediated VEGFR-2 signaling was comparable to that inhibited by aflibercept or bevacizumab. See FIG. 9.

### Example 7: Comparison of the Effect of EB-101 with Aflibercept on Inhibition of VEGF-C Mediated VEGFR2 Signaling in VEGFR2-NFAT-RE Luciferase Reporter Cells

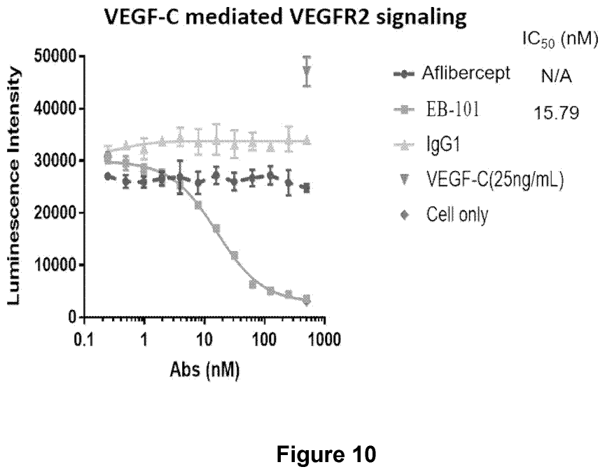

Recombinant human VEGF-C were mixed with serial dilutions of the test compound, incubated at room temperature for 30 minutes, and then added into wells containing 4×10⁴ VEGFR-2 (KDR)-NFAT-RE luciferase reporter cells/well followed by an incubation at 37° C. for 6 hours. Luciferase signal was detected by a plate reader after addition of 50 μl of Bright-Lite. EB-101, but not aflibercept, had concentration-dependent inhibition of VEGF-C mediated VEGFR-2 signaling. See FIG. 10.

### Example 8: Comparison of EB-101 with Aflibercept and a Recombinant Human VEGFR-3 Containing 7 Extracellular Domains (rhVEGFR-3-7ECDs) on their Inhibitory Effect on VEGF-C Mediated VEGFR-2 Signaling in VEGFR-2-NFAT-RE Luciferase Reporter Cells

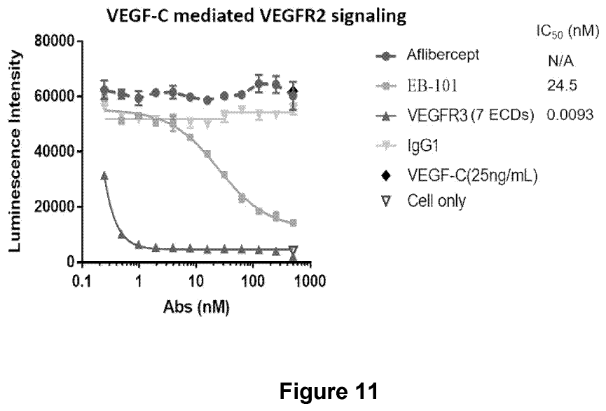

Recombinant human VEGF-C were mixed with serial dilutions of the test compound, incubated at room temperature for 30 minutes, and then added into wells containing 4×10⁴ VEGFR-2 (KDR)-NFAT-RE luciferase reporter cells/well followed by an incubation at 37° C. for 6 hours. Luciferase signal was detected by a plate reader after addition of 50 μl of Bright-Lite. EB-101 and rhVEGFR-3, but not aflibercept, had dose-dependent inhibition of VEGF-C mediated VEGFR-2 signaling. See FIG. 11.

### Example 9: Quantitative Analyses of the Effect of EB-101 with Aflibercept and a Recombinant Human VEGFR-3 Containing 7 Extracellular Domains (rhVEGFR-3) on Inhibition of VEGFR-2 Signaling in VEGFR-2-NFAT-RE Luciferase Reporter Cells Stimulated by VEGF-A₁₆₅ and VEGF-C

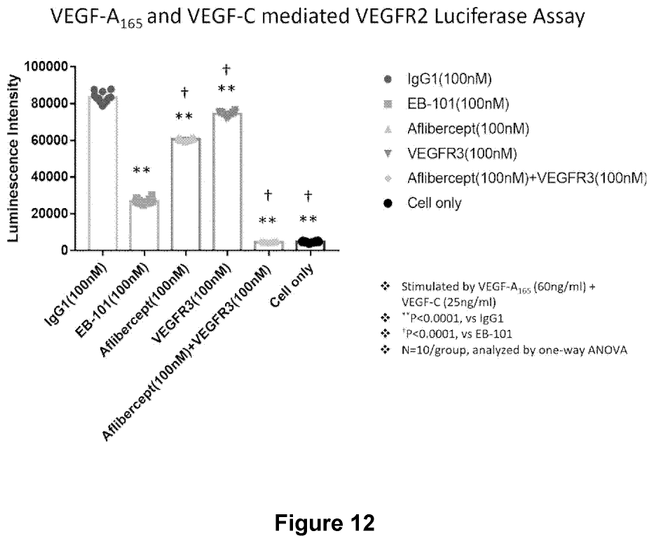

The test compound was mixed with a cocktail containing 60 ng/ml of VEGF-A₁₆₅ and 25 ng/ml of VEGF-C and incubated at room temperature for 30 minutes and then the resultant solution was added into wells containing 4×10⁴ VEGFR-2-NFAT-RE luciferase reporter cells/well, followed by an incubation at 37° C. for 6 hours. Luciferase signal was detected by a plate reader after addition of 50 μl of Bright-Lite. EB-101 and a combination of aflibercept with rhVEGFR-3 effectively inhibited VEGF-A₁₆₅ plus VEGF-C mediated VEGFR-2 signaling while either aflibercept or rhVEGFR3 alone showed only limited effect on inhibition of VEGF-A₁₆₅ and VEGF-C-mediated VEGFR-2 signaling. See FIG. 12.

### Example 10: Comparison of the Effect of EB-101 with Aflibercept and Bevacizumab on VEGF-A₁₆₅-Mediated Proliferation of Human Lymphatic Endothelial Cells (HLECs)

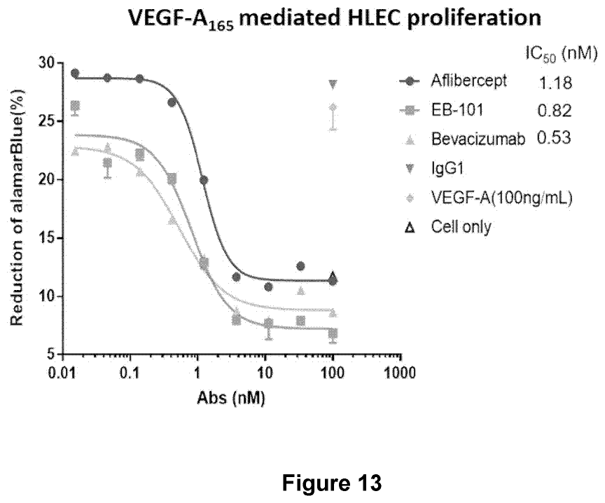

Primary HLECs were seeded into 96-well plates at a density of 2500 cells/well and cultured at 37° C. for overnight. On the next day, recombinant human VEGF-A₁₆₅ was mixed with serial dilutions of the test compound and incubate at room temperature for 30 minutes, and then the resultant solution was added into each well followed by an incubation at 37° C. for 72 hours. Cell proliferation was evaluated by addition of 10% AlamarBlue dye into each well and incubated for 5 hours followed by fluorescent reading using a plate reader. The effect of EB-101 on inhibiting VEGF-A₁₆₅-mediated HLEC proliferation was comparable to that inhibited by aflibercept and bevacizumab. See FIG. 13.

### Example 11: Comparison of the Effect of EB-101 with Aflibercept and a Recombinant Human VEGFR-3 Containing 7 Extracellular Domains (rhVEGFR-3 ECDs) on VEGF-C Mediated HLEC Proliferation

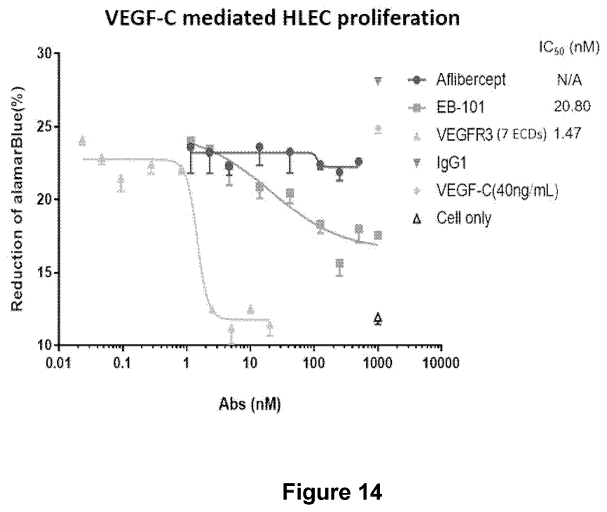

Primary HLECs were seeded into 96-well plates at a density of 2500 cells/well and cultured in an incubator at 37° C. for overnight. On the next day, recombinant human VEGF-C were mixed with serial dilutions of the test compound and incubate at room temperature for 30 minutes, and then the resultant solution was added into each well followed by an incubation at 37° C. for 72 hours. Cell proliferation was evaluated by addition of 10% AlamarBlue dye into each well and incubated for 5 hours followed by fluorescent reading using a plate reader. EB-101 and rhVEGFR-3, but not aflibercept, showed concentration-dependent inhibition of VEGF-C mediated HLEC proliferation. See FIG. 14.

### Example 12: Quantitative Analyses of EB-101, Aflibercept and a Recombinant Human VEGFR-3 Containing 7 Extracellular Domains (rhVEGFR-3 ECDs), on VEGF-A₁₆₅ and VEGF-C Mediated Human Lymphatic Endothelial Cell (HLEC) Proliferation

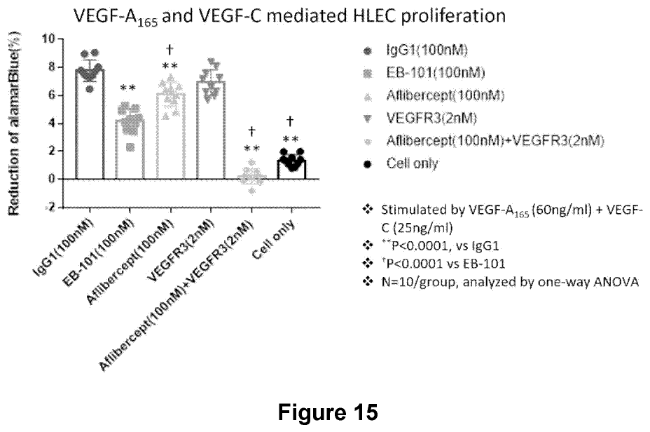

Primary HLECs were seeded into 96-well plates at a density of 2500 cells/well and cultured in an incubator at 37° C. for overnight. On the next day, a cocktail containing 60 ng/ml of VEGF-A₁₆₅ and 25 ng/ml of VEGF-C were mixed with the test compound and incubated at room temperature for 30 minutes, and then the resultant solution was added into wells followed by an incubation at 37° C. for 72 hours. Cell proliferation was evaluated by addition of 10% AlamarBlue dye and incubated for 5 hours followed by fluorescent reading using a plate reader. EB-101 and the combination of aflibercept with rhVEGFR-3 effectively inhibited VEGF-A₁₆₅ and VEGF-C mediated VEGFR2 signaling, while either aflibercept or rhVEGFR3 alone had only limited inhibition in VEGF-A₁₆₅ and VEGF-C mediated HLEC proliferation. See FIG. 15.

### Example 13: Molecular Design and Expression of EB-101BIb

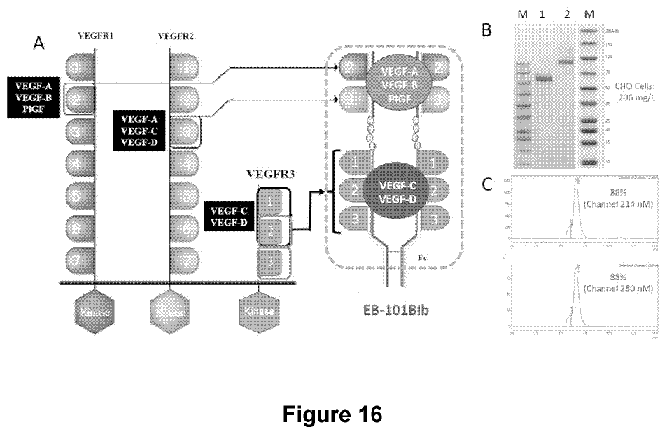

EB-101BIb is a recombinant human Fc fusion protein including the binding domain 2 of VEGFR-1, domain 3 of VEGFR-2, and domains 1-3 of VEGFR-3. Gene encoding the amino acid sequence of EB-101BIb as indicated by the SEQ ID NO: 16 was synthetized and a vector for expressing the protein was constructed. The expression vector was used to transiently transfect CHO cells with chemically defined culture media. The produced protein was purified by Protein A affinity column, ultrafiltration and then subjected to 0.2 μm sterile filtration to get the bulk of high purity. Expression of EB-101BIb in CHO cells produced a protein with a MW ˜200KDa (non-reduced form at SDS-PAGE) and a purity of 88% when analyzed by SEC-HPLC. See FIG. 16.

### Example 14: Comparison of the Effect of EB-101BIb with Aflibercept, EB-101, EB-101BIa on VEGF-A Mediated VEGFR-2 Signaling in VEGFR2-NFAT-RE Luciferase Reporter Cells

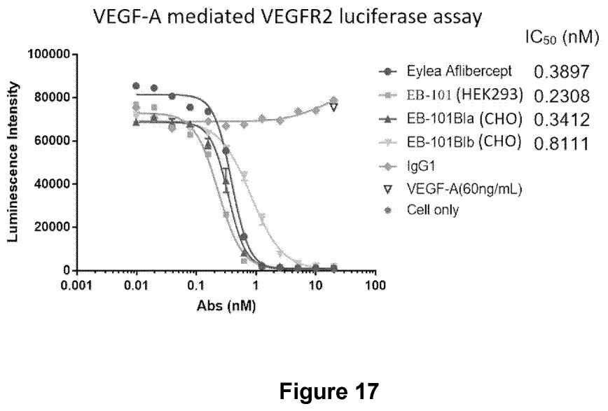

Recombinant human VEGF-A 165 were mixed with serial dilutions of the test compound, incubated at room temperature for 30 minutes, and then added into wells containing 4×10⁴ VEGFR2 (KDR)-NFAT-RE luciferase reporter cells/well followed by an incubation at 37° C. for 6 hours. Luciferase signal was detected by a plate reader after addition of 50 μl of Bright-Lite. The effect of EB-101BIb on inhibiting VEGF-A 165 mediated VEGFR-2 signaling was comparable to that inhibited by aflibercept, EB-101 and EB-101BIa. The latter two molecules contain binding domain 2 of VEGFR-1, domain 3 of VEGFR-2 and domains 1-2 but not domain 3 of VEGFR-3 and were expressed in HEK293 (EB-101) and CHO (EB-101BIa) cells, respectively. See FIG. 17.

### Example 15: Comparison of the Effect of EB-101BIb with Aflibercept, EB-101 and EB-101BIa on VEGF-C and -D Mediated VEGFR-2 Signaling in VEGFR-2-NFAT-RE Luciferase Reporter Cells

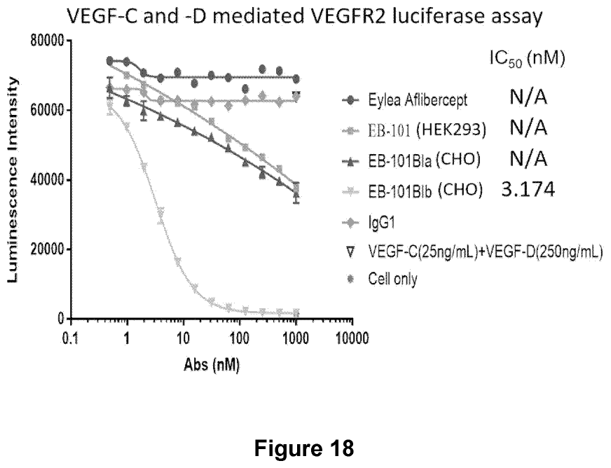

Recombinant human VEGF-C and -D were mixed with serial dilutions of the test compound, incubated at room temperature for 30 minutes, and then added into wells containing 4×10⁴ VEGFR-2 (KDR)-NFAT-RE luciferase reporter cells/well followed by an incubation at 37° C. for 6 hours. Luciferase signal was detected by a plate reader after addition of 50 μl of Bright-Lite. EB-101BIb showed a concentration-dependent inhibition of VEGF-C and -D mediated VEGFR-2 signaling, while EB-101 and EB-101BIa showed limited inhibition of VEGF-C and -D mediated VEGFR-2 signaling. Aflibercept showed no effect on VEGF-C and -D mediated VEGFR-2 signaling. EB-101 and EB-101BIa contain the binding domain 2 of VEGFR-1, domain 3 of VEGFR-2 and domains 1-2 but not domain 3 of VEGFR-3 and were expressed in HEK293 (EB-101) and CHO (EB-101BIa) cells, respectively. See FIG. 18.

### Example 16: Comparison of the Effect of EB-101BIb with Aflibercept, EB-101 and a Combination of Aflibercept with a Recombinant Human VEGFR-3 on VEGF-A, -C and -D Mediated VEGFR-2 Signaling in VEGFR-2-NFAT-RE Luciferase Reporter Cells

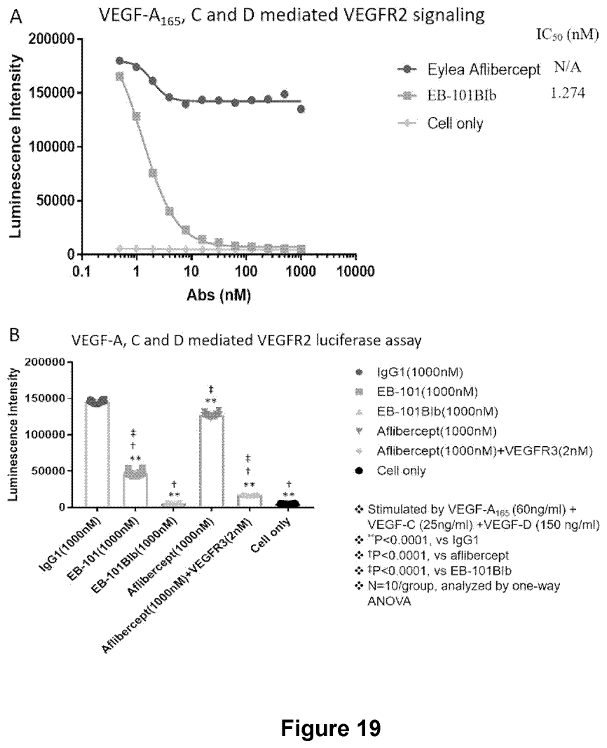

The test compound was mixed with a cocktail containing 60 ng/ml of VEGF-A₁₆₅ and 25 ng/ml of VEGF-C and 150 ng/ml of VEGF-D followed by incubation at room temperature for 30 minutes, and then the resultant solution was added into wells containing 4×10⁴ VEGFR-2-NFAT-RE luciferase reporter cells/well, followed by an incubation at 37° C. for 6 hours. Luciferase signal was detected by a plate reader after addition of 50 μl of Bright-Lite. Aflibercept showed little inhibition of VEGFR-2 signaling while EB-101BIb showed concentration-dependent inhibition of VEGFR-2 signaling in VEGFR-2-NFAT-RE luciferase reporter cells stimulated by a cocktail consisting of VEGF-A₁₆₅, -C and -D (FIG. 19A). EB-101BIb completely inhibited VEGF-A₁₆₅, VEGF-C and VEGF-D mediated VEGFR-2 signaling. EB-101 showed limited inhibitory effect while aflibercept had the least inhibition of VEGF-A, -C, and -D mediated VEGFR-2 signaling compared with EB-101BIb and EB-101. The complete inhibition effected EB-101BIb was replicated by a combination of EB-101 with the recombinant human VEGFR-3 (FIG. 19B). EB-101 contains the binding domain 2 of VEGFR-1, domain 3 of VEGFR-2 and domains 1-2 but not domain 3 of VEGFR-3. EB-101BIb contains the binding domain 2 of VEGFR-1, domain 3 of VEGFR-2, and domains 1-3 of VEGFR-3. See FIG. 19.

### Example 17: Comparison of the Effect of EB-101BIb with Aflibercept and EB-101BIa on VEGF-A₁₆₅-Mediated Proliferation of Human Lymphatic Endothelial Cells (HLECs)

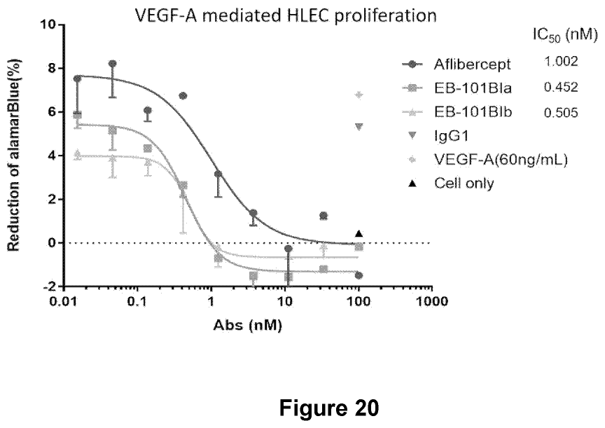

Primary HLECs were seeded into 96-well plates at a density of 2500 cells/well and cultured at 37° C. for overnight. On the next day, recombinant human VEGF-A₁₆₅ was mixed with serial dilutions of the test compound and incubate at room temperature for 30 minutes, and then the resultant solution was added into each well followed by an incubation at 37° C. for 72 hours. Cell proliferation was evaluated by addition of 10% AlamarBlue dye into each well and incubated for 5 hours followed by fluorescent reading using a plate reader. The effect of EB-101BIb on inhibiting VEGF-A₁₆₅-mediated HLEC proliferation was comparable to that inhibited by aflibercept and EB-101BIa. See FIG. 20.

### Example 18: Comparison of the Effect of EB-101BIb with Aflibercept, EB-101BIa and a Recombinant Human VEGFR-3 Containing 7 Extracellular Domains on VEGF-C and VEGF-D Mediated HLEC Proliferation

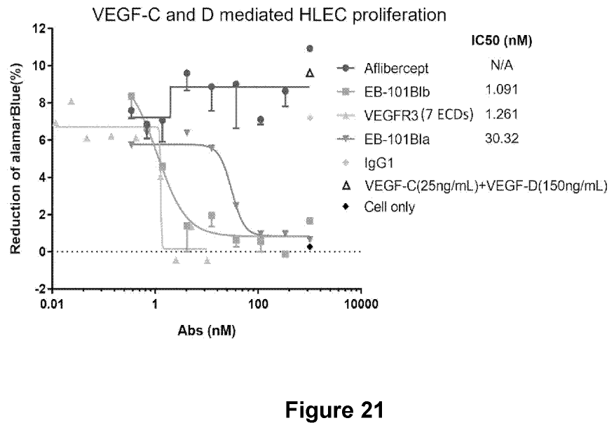

Primary HLECs were seeded into 96-well plates at a density of 2500 cells/well and cultured in an incubator at 37° C. for overnight. On the next day, recombinant human VEGF-C (25 ng/ml) and VEGF-D (150 ng/ml) were mixed with serial dilutions of the test compound and incubated at room temperature for 30 minutes, and then the resultant solution was added into each well followed by an incubation at 37° C. for 72 hours. Cell proliferation was evaluated by addition of 10% AlamarBlue dye into each well and incubated for 5 hours followed by fluorescent reading using a plate reader. EB-101BIb and rhVEGFR-3 showed more potent effect on inhibition of VEGF-C and VEGF-D mediated HLEC proliferation compared with EB-101Bia treated group. See FIG. 21.

### Example 19: Comparison of the Effect of EB-101BIb with Aflibercept, a Recombinant Human VEGFR-3 Containing 7 Extracellular Domains (7 ECDs) and a Combination of Aflibercept with the VEGFR-3 on VEGF-A, -C and -D Mediated Human Lymphatic Endothelial Cell (HLEC) Proliferation

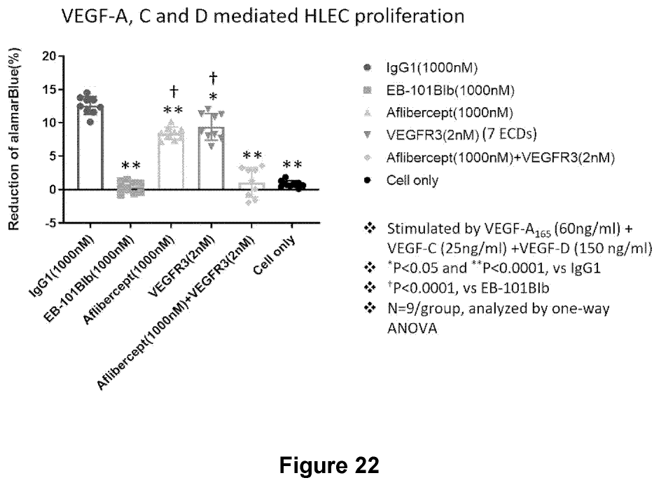

Primary HLECs were seeded into 96-well plates at a density of 2500 cells/well and cultured in an incubator at 37° C. for overnight. On the next day, a cocktail containing recombinant human VEGF-A₁₆₅, VEGF-C and VEGF-D was mixed with equal volume of serial dilutions of the test compound and incubated at room temperature for 30 minutes (final concentrations of VEGF-A₁₆₅, -C and -D: 60, 25 and 150 ng/ml, respectively). The resultant solutions were added into 9 wells in each group and then incubated at 37° C. for 72 hours. Cell proliferation was evaluated by addition of 10% AlamarBlue dye into each well and incubated for 5 hours followed by fluorescent reading using a plate reader. Either aflibercept or VEGFR-3 alone showed limited inhibition while EB-101BIb or a combination of aflibercept with VEGFR-3 completely inhibited HLEC proliferation stimulated by VEGF-A₁₆₅, -C and -D. See FIG. 22.

NUCLEIC ACID AND AMINO ACID SEQUENCE LISTING
(SEQ ID NO: 1)
ag tgataccggt agacctttcg tagagatgta cagtgaaatc cccgaaatta tacacatgac
tgaaggaagg gagctcgtca ttccctgccg ggttacgtca cctaacatca ctgttacttt
aaaaaagttt ccacttgaca ctttgatccc tgatggaaaa cgcataatct gggacagtag
aaagggcttc atcatatcaa atgcaacgta caaagaaata gggcttctga cctgtgaagc
aacagtcaat gggcatttgt ataagacaaa ctatctcaca catcgacaaa ccaatacaat cata
(SEQ ID NO: 2)
SDTGRPFVEMYSEIPEIIHMTEGRELVIPCRVTSPNITVTLKKFPLDTLIPDGKR
IIWDSRKGFIISNATYKEIGLLTCEATVNGHLYKTNYLTHRQTNTII
(SEQ ID NO: 3)
gatgtg gttctgagtc cgtctcatgg aattgaacta tctgttggag aaaagcttgt
cttaaattgt acagcaagaa ctgaactaaa tgtggggatt gacttcaact gggaataccc
ttcttcgaag catcagcata agaaacttgt aaaccgagac ctaaaaaccc agtctgggag
tgagatgaag aaatttttga gcaccttaac tatagatggt gtaacccgga gtgaccaagg
attgtacacc tgtgcagcat ccagtgggct gatgaccaag aagaacagca catttgtcag
ggtccatgaa aag
(SEQ ID NO: 4)
DVVLSPSHGIELSVGEKLVLNCTARTELNVGIDENWEYPSSKHQHKKLVNRD
LKTQSGSEMKKFLSTLTIDGVTRSDQGLYTCAASSGLMTKKNSTFVRVHEK
(SEQ ID NO: 5)
tac tcc atg acc ccc ccg acc ttg aac atc acg gag gag tca cac gtc
atc gac acc ggt gac agccctg tcc atc tcc tgc agg gga cag cac ccc
ctc gag tgg gct tgg cca gga gct cag gag gcg cca gcc acc gga gac
aag gac agc gag gac acg ggg gtg gtg cga gac tgc gag ggc aca gac
gcc agg ccc tac tgc aag gtg ttg ctg ctg cac gag gta cat gcc aac
gac aca ggc agc tac gtc tgc tac tac aag tac atc aag gca cgc atc
gag ggc acc acg gcc gcc agc tcc tac gtg ttc gtg aga gac ttt gag
cag cca ttc atc aac aag cct gac acg ctc ttg gtc aac agg aag gac
gcc atg tgg gtg ccc tgt ctg gtg tcc atc ccc ggc ctc aat gtc acg
ctg cgc tcg caa agc tcg gtg ctg tgg cca gac ggg cag gag gtg gtg
tgg gat gac cgg cgg ggc atg ctc gtg tcc acg cca ctg ctg cac gat
gcc ctg tac ctg cag tgc gag acc acc tgg gga gac cag gac ttc ctt
tcc aac ccc ttc ctg gtg cac atc aca ggc aac gag ct
(SEQ ID NO: 6)
YSMTPPTLNITEESHVIDTGDSLSISCRGQHPLEWAWPGAQEAPATGDKDSE
DTGVVRDCEGTDARPYCKVLLLHEVHANDTGSYVCYYKYIKARIEGTTAASSYVFVRD
FEQPFINKPDTLLVNRKDAMWVPCLVSIPGLNVTLRSQSSVLWPDGQEVVWDDRRGML
VSTPLLHDALYLQCETTWGDQDFLSNPFLVHITGNEL
(SEQ ID NO: 7)
gacaaaa ctcacacatg cccaccgtgc ccagcacctg aactcctggg gggaccgtca
gtcttcctct tccccccaaa acccaaggac accctcatga tctcccggac ccctgaggtc
acatgcgtgg tggtggacgt gagccacgaa gaccctgagg tcaagttcaa ctggtacgtg
gacggcgtgg aggtgcataa tgccaagaca aagccgcggg aggagcagta caacagcacg
taccgtgtgg tcagcgtcct caccgtcctg caccaggact ggctgaatgg caaggagtac
aagtgcaagg tctccaacaa agccctccca gcccccatcg agaaaaccat ctccaaagcc
aaagggcagc cccgagaacc acaggtgtac accctgcccc catcccggga tgagctgacc
aagaaccagg tcagcctgac ctgcctggtc aaaggcttct atcccagcga catcgccgtg
gagtgggaga gcaatgggca gccggagaac aactacaaga ccacgcctcc cgtgctggac
tccgacggct ccttcttcct ctacagcaag ctcaccgtgg acaagagcag gtggcagcag
gggaacgtct tctcatgctc cgtgatgcat gaggctctgc acaaccacta cacgcagaag
agcctctccc tgtctccggg t
(SEQ ID NO: 8)
DKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPG
(SEQ ID NO: 9)
ggtggctctg gagggggttc cggtggcgga ggttcaggtg gcggaggggg ttcgggtggc
ggaggg
(SEQ ID NO: 10)
GGSGG GSGGG GSGGG GGSGG GG
ag tgataccggt agacctttcg tagagatgta cagtgaaatc cccgaaatta tacacatgac
tgaaggaagg gagctcgtca ttccctgccg ggttacgtca cctaacatca ctgttacttt
aaaaaagttt ccacttgaca ctttgatccc tgatggaaaa cgcataatct gggacagtag
aaagggcttc atcatatcaa atgcaacgta caaagaaata gggcttctga cctgtgaagc
aacagtcaat gggcatttgt ataagacaaa ctatctcaca catcgacaaa ccaatacaat cata
gatgtg gttctgagtc cgtctcatgg aattgaacta tctgttggag aaaagcttgt
cttaaattgt acagcaagaa ctgaactaaa tgtggggatt gacttcaact gggaataccc
ttcttcgaag catcagcata agaaacttgt aaaccgagac ctaaaaaccc agtctgggag
tgagatgaag aaatttttga gcaccttaac tatagatggt gtaacccgga gtgaccaagg
attgtacacc tgtgcagcat ccagtgggct gatgaccaag aagaacagca catttgtcag
ggtccatgaa aag
tac tcc atg acc ccc ccg acc ttg aac atc acg gag gag tca cac gtc
atc gac acc ggt gac agc ctg tcc atc tcc tgc agg gga cag cac ccc
ctc gag tgg gct tgg cca gga gct cag gag gcg cca gcc acc gga gac
aag gac agc gag gac acg ggg gtg gtg cga gac tgc gag ggc aca gac
gcc agg ccc tac tgc aag gtg ttg ctg ctg cac gag gta cat gcc aac
gac aca ggc agc tac gtc tgc tac tac aag tac atc aag gca cgc atc
gag ggc acc acg gcc gcc agc tcc tac gtg ttc gtg aga gac ttt gag
cag cca ttc atc aac aag cct gac acg ctc ttg gtc aac agg aag gac
gcc atg tgg gtg ccc tgt ctg gtg tcc atc ccc ggc ctc aat gtc acg
ctg cgc tcg caa agc tcg gtg ctg tgg cca gac ggg cag gag gtg gtg
tgg gat gac cgg cgg ggc atg ctc gtg tcc acg cca ctg ctg cac gat
gcc ctg tac ctg cag tgc gag acc acc tgg gga gac cag gac ttc ctt
tcc aac ccc ttc ctg gtg cac atc aca ggc aac gag ctc
ggtggctctg gagggggttc cggtggcgga ggttcaggtg gcggaggggg ttcgggtggc
ggaggg
(SEQ ID NO: 11)
gacaaaa ctcacacatg cccaccgtgc ccagcacctg aactcctggg gggaccgtca
gtcttcctct tccccccaaa acccaaggac accctcatga tctcccggac ccctgaggtc
acatgcgtgg tggtggacgt gagccacgaa gaccctgagg tcaagttcaa ctggtacgtg
gacggcgtgg aggtgcataa tgccaagaca aagccgcggg aggagcagta caacagcacg
taccgtgtgg tcagcgtcct caccgtcctg caccaggact ggctgaatgg caaggagtac
aagtgcaagg tctccaacaa agccctccca gcccccatcg agaaaaccat ctccaaagcc
aaagggcagc cccgagaacc acaggtgtac accctgcccc catcccggga tgagctgacc
aagaaccagg tcagcctgac ctgcctggtc aaaggcttct atcccagcga catcgccgtg
gagtgggaga gcaatgggca gccggagaac aactacaaga ccacgcctcc cgtgctggac
tccgacggct ccttcttcct ctacagcaag ctcaccgtgg acaagagcag gtggcagcag
gggaacgtct tctcatgctc cgtgatgcat gaggctctgc acaaccacta cacgcagaag
agcctctccc tgtctccggg t
(SEQ. ID. NO. 12)
SDTGRPFVEMYSEIPEIIHMTEGRELVIPCRVTSPNITVTLKKFPLDTLIPDGKR
IIWDSRKGFIISNATYKEIGLLTCEATVNGHLYKTNYLTHRQTNTIIDVVLSPSHGIELSVG
EKLVLNCTARTELNVGIDENWEYPSSKHQHKKLVNRDLKTQSGSEMKKFLSTLTIDGVT
RSDQGLYTCAASSGLMTKKNSTFVRVHEKYSMTPPTLNITEESHVIDTGDSLSISCRGQH
PLEWAWPGAQEAPATGDKDSEDTGVVRDCEGTDARPYCKVLLLHEVHANDTGSYVCY
YKYIKARIEGTTAASSYVFVRDFEQPFINKPDTLLVNRKDAMWVPCLVSIPGLNVTLRSQ
SSVLWPDGQEVVWDDRRGMLVSTPLLHDALYLQCETTWGDQDFLSNPFLVHITGNELG
GSGGGSGGGGSGGGGGSGGGGDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEAL
HNHYTQKSLSLSPG
(SEQ ID NO: 13)
MQRGAALCLRLWLCLGLLDGLVSGYSMTPPTLNITEESHVIDTGDSLSISCR
GQHPLEWAWPGAQEAPATGDKDSEDTGVVRDCEGTDARPYCKVLLLHEVHANDTGSY
VCYYKYIKARIEGTTAASSYVFVRDFEQPFINKPDTLLVNRKDAMWVPCLVSIPGLNVT
LRSQSSVLWPDGQEVVWDDRRGMLVSTPLLHDALYLQCETTWGDQDFLSNPFLVHITG
NELYDIQLLPRKSLELLVGEKLVLNCTVWAEFNSGVTFDWDYPGKQAERGKWVPERRS
QQTHTELSSILTIHNVSQHDLGSYVCKANNGIQRFRESTEVIVHENPFISVEWLKGPILEA
TAGDELVKLPVKLAAYPPPEFQWYKDGKALSGRHSPHALVLKEVTEASTGTYTLALWN
SAAGLRRNISLELVVNVPPQIHEKEASSPSIYSRHSRQALTCTAYGVPLPLSIQWHWRPW
TPCKMFAQRSLRRRQQQDLMPQCRDWRAVTTQDAVNPIESLDTWTEFVEGKNKTVSK
LVIQNANVSAMYKCVVSNKVGQDERLIYFYVTTIPDGFTIESKPSEELLEGQPVLLSCQA
DSYKYEHLRWYRLNLSTLHDAHGNPLLLDCKNVHLFATPLAASLEEVAPGARHATLSL
SIPRVAPEHEGHYVCEVQDRRSHDKHCHKKYLSVQALEAPRLTQNLTDLLVNVSDSLE
MQCLVAGAHAPSIVWYKDERLLEEKSGVDLADSNQKLSIQRVREEDAGRYLCSVCNAK
GCVNSSASVAVEGSEDKGSMEIVILVGTGVIAVFFWVLLLLIFCNMRRPAHADIKTGYLS
IIMDPGEVPLEEQCEYLSYDASQWEFPRERLHLGRVLGYGAFGKVVEASAFGIHKGSSC
DTVAVKMLKEGATASEHRALMSELKILIHIGNHLNVVNLLGACTKPQGPLMVIVEFCKY
GNLSNFLRAKRDAFSPCAEKSPEQRGRFRAMVELARLDRRRPGSSDRVLFARFSKTEGG
ARRASPDQEAEDLWLSPLTMEDLVCYSFQVARGMEFLASRKCIHRDLAARNILLSESDV
VKICDFGLARDIYKDPDYVRKGSARLPLKWMAPESIFDKVYTTQSDVWSFGVLLWEIFS
LGASPYPGVQINEEFCQRLRDGTRMRAPELATPAIRRIMLNCWSGDPKARPAFSELVEIL
GDLLQGRGLQEEEEVCMAPRSSQSSEEGSFSQVSTMALHIAQADAEDSPPSLQRHSLAA
RYYNWVSFPGCLARGAETRGSSRMKTFEEFPMTPTTYKGSVDNQTDSGMVLASEEFEQI
ESRHRQESGFSCKGPGQNVAVTRAHPDSQGRRRRPERGARGGQVFYNSEYGELSEPSEE
DHCSPSARVTFFTDNSY
(SEQ ID NO: 14)
YSMTPPTLNITEESHVIDTGDSLSISCRGQHPLEWAWPGAQEAPATGDKDSE
DTGVVRDCEGTDARPYCKVLLLHEVHANDTGSYVCYYKYIKARIEGTTAAS
(SEQ ID NO: 15)
YSMTPPTLNITEESHVIDTGDSLSISCRGQHPLEWAWPGAQEAPATGDKDSE
DTGVVRDCEGTDARPYCKVLLLHEVHANDTGSYVCYYKYIKARIEGTTAASSYVFVRD
FEQPFINKPDTLLVNRKDAMWVPCLVSIPGLNVTLRSQSSVLWPDGQEVVWDDRRGML
VSTPLLHDALYLQCETTWGDQDFLSNPFLVHITGNELYDIQLLPRKSLELLVGEKLVLNC
TVWAEFNSGVTFDWDYPGKQAERGKWVPERRSQQTHTELSSILTIHNVSQHDLGSYVC
KANNGIQRFRESTEVIVHEN
(SEQ ID NO: 16)
SDTGRPFVEMYSEIPEIIHMTEGRELVIPCRVTSPNITVTLKKFPLDTLIPDGKR
IIWDSRKGFIISNATYKEIGLLTCEATVNGHLYKTNYLTHRQTNTIIDVVLSPSHGIELSVG
EKLVLNCTARTELNVGIDENWEYPSSKHQHKKLVNRDLKTQSGSEMKKFLSTLTIDGVT
RSDQGLYTCAASSGLMTKKNSTFVRVHEKGGSGGGSGGGGSGGGGGSGGGGYSMTPP
TLNITEESHVIDTGDSLSISCRGQHPLEWAWPGAQEAPATGDKDSEDTGVVRDCEGTDA
RPYCKVLLLHEVHANDTGSYVCYYKYIKARIEGTTAASSYVFVRDFEQPFINKPDTLLV
NRKDAMWVPCLVSIPGLNVTLRSQSSVLWPDGQEVVWDDRRGMLVSTPLLHDALYLQ
CETTWGDQDFLSNPFLVHITGNELYDIQLLPRKSLELLVGEKLVLNCTVWAEFNSGVTF
DWDYPGKQAERGKWVPERRSQQTHTELSSILTIHNVSQHDLGSYVCKANNGIQRFREST
EVIVHENDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPG

```
SEQUENCE LISTING
<110> Eluminex Biosciences Ltd.
<120> Inhibitors of Angiogenic Factors
<130> EluminexBiosciences-01
<160> 16
<170> PatentIn version 3.5
<210> 1
<211> 306
<212> DNA
<213> Homo sapiens
<400> 1
agtgataccg gtagaccttt cgtagagatg tacagtgaaa tccccgaaat tatacacatg 60
actgaaggaa gggagctcgt cattccctgc cgggttacgt cacctaacat cactgttact 120
ttaaaaaagt ttccacttga cactttgatc cctgatggaa aacgcataat ctgggacagt 180
agaaagggct tcatcatatc aaatgcaacg tacaaagaaa tagggcttct gacctgtgaa 240
gcaacagtca atgggcattt gtataagaca aactatctca cacatcgaca aaccaataca 300
atcata 306
<210> 2
<211> 102
<212> PRT
<213> Homo sapiens
<400> 2
Ser Asp Thr Gly Arg Pro Phe Val Glu Met Tyr Ser Glu Ile Pro Glu
1 5 10 15
Ile Ile His Met Thr Glu Gly Arg Glu Leu Val Ile Pro Cys Arg Val
                        20 25 30
Thr Ser Pro Asn Ile Thr Val Thr Leu Lys Lys Phe Pro Leu Asp Thr
                35 40 45
Leu Ile Pro Asp Gly Lys Arg Ile Ile Trp Asp Ser Arg Lys Gly Phe
        50 55 60
Ile Ile Ser Asn Ala Thr Tyr Lys Glu Ile Gly Leu Leu Thr Cys Glu
65 70 75 80
Ala Thr Val Asn Gly His Leu Tyr Lys Thr Asn Tyr Leu Thr His Arg
                                85 90 95
Gln Thr Asn Thr Ile Ile
                        100
<210> 3
<211> 309
<212> DNA
<213> Homo sapiens
<400> 3
gatgtggttc tgagtccgtc tcatggaatt gaactatctg ttggagaaaa gcttgtctta 60
aattgtacag caagaactga actaaatgtg gggattgact tcaactggga atacccttct 120
tcgaagcatc agcataagaa acttgtaaac cgagacctaa aaacccagtc tgggagtgag 180
atgaagaaat ttttgagcac cttaactata gatggtgtaa cccggagtga ccaaggattg 240
tacacctgtg cagcatccag tgggctgatg accaagaaga acagcacatt tgtcagggtc 300
catgaaaag 309
<210> 4
<211> 103
<212> PRT
<213> Homo sapiens
<400> 4
Asp Val Val Leu Ser Pro Ser His Gly Ile Glu Leu Ser Val Gly Glu
1 5 10 15
Lys Leu Val Leu Asn Cys Thr Ala Arg Thr Glu Leu Asn Val Gly Ile
                        20 25 30
Asp Phe Asn Trp Glu Tyr Pro Ser Ser Lys His Gln His Lys Lys Leu
                35 40 45
Val Asn Arg Asp Leu Lys Thr Gln Ser Gly Ser Glu Met Lys Lys Phe
        50 55 60
Leu Ser Thr Leu Thr Ile Asp Gly Val Thr Arg Ser Asp Gln Gly Leu
65 70 75 80
Tyr Thr Cys Ala Ala Ser Ser Gly Leu Met Thr Lys Lys Asn Ser Thr
                                85 90 95
Phe Val Arg Val His Glu Lys
                        100
<210> 5
<211> 614
<212> DNA
<213> Homo sapiens
<400> 5
tactccatga cccccccgac cttgaacatc acggaggagt cacacgtcat cgacaccggt 60
gacagcctgt ccatctcctg caggggacag caccccctcg agtgggcttg gccaggagct 120
caggaggcgc cagccaccgg agacaaggac agcgaggaca cgggggtggt gcgagactgc 180
gagggcacag acgccaggcc ctactgcaag gtgttgctgc tgcacgaggt acatgccaac 240
gacacaggca gctacgtctg ctactacaag tacatcaagg cacgcatcga gggcaccacg 300
gccgccagct cctacgtgtt cgtgagagac tttgagcagc cattcatcaa caagcctgac 360
acgctcttgg tcaacaggaa ggacgccatg tgggtgccct gtctggtgtc catccccggc 420
ctcaatgtca cgctgogctc gcaaagctcg gtgctgtggc cagacgggca ggaggtggtg 480
tgggatgacc ggcggggcat gctcgtgtcc acgccactgc tgcacgatgc cctgtacctg 540
cagtgcgaga ccacctgggg agaccaggac ttcctttcca accccttcct ggtgcacatc 600
acaggcaacg agct 614
<210> 6
<211> 205
<212> PRT
<213> Homo sapiens
<400> 6
Tyr Ser Met Thr Pro Pro Thr Leu Asn Ile Thr Glu Glu Ser His Val
1 5 10 15
Ile Asp Thr Gly Asp Ser Leu Ser Ile Ser Cys Arg Gly Gln His Pro
                        20 25 30
Leu Glu Trp Ala Trp Pro Gly Ala Gln Glu Ala Pro Ala Thr Gly Asp
                35 40 45
Lys Asp Ser Glu Asp Thr Gly Val Val Arg Asp Cys Glu Gly Thr Asp
        50 55 60
Ala Arg Pro Tyr Cys Lys Val Leu Leu Leu His Glu Val His Ala Asn
65 70 75 80
Asp Thr Gly Ser Tyr Val Cys Tyr Tyr Lys Tyr Ile Lys Ala Arg Ile
                                85 90 95
Glu Gly Thr Thr Ala Ala Ser Ser Tyr Val Phe Val Arg Asp Phe Glu
                        100 105 110
Gln Pro Phe Ile Asn Lys Pro Asp Thr Leu Leu Val Asn Arg Lys Asp
                115 120 125
Ala Met Trp Val Pro Cys Leu Val Ser Ile Pro Gly Leu Asn Val Thr
        130 135 140
Leu Arg Ser Gln Ser Ser Val Leu Trp Pro Asp Gly Gln Glu Val Val
145 150 155 160
Trp Asp Asp Arg Arg Gly Met Leu Val Ser Thr Pro Leu Leu His Asp
                              165 170 175
Ala Leu Tyr Leu Gln Cys Glu Thr Thr Trp Gly Asp Gln Asp Phe Leu
                        180 185 190
Ser Asn Pro Phe Leu Val His Ile Thr Gly Asn Glu Leu
                195 200 205
<210> 7
<211> 678
<212> DNA
<213> Homo sapiens
<400> 7
gacaaaactc acacatgccc accgtgccca gcacctgaac toctgggggg accgtcagtc 60
ttcctcttcc ccccaaaacc caaggacacc ctcatgatct cccggacccc tgaggtcaca 120
tgcgtggtgg tggacgtgag ccacgaagac cctgaggtca agttcaactg gtacgtggac 180
ggcgtggagg tgcataatgc caagacaaag ccgcgggagg agcagtacaa cagcacgtac 240
cgtgtggtca gcgtcctcac cgtcctgcac caggactggc tgaatggcaa ggagtacaag 300
tgcaaggtct ccaacaaagc cctoccagcc cccatcgaga aaaccatctc caaagccaaa 360
gggcagcccc gagaaccaca ggtgtacacc ctgcccccat cccgggatga gctgaccaag 420
aaccaggtca gcctgacctg cctggtcaaa ggcttctatc ccagcgacat cgccgtggag 480
tgggagagca atgggcagcc ggagaacaac tacaagacca cgcctcccgt gctggactcc 540
gacggctcct tcttcctcta cagcaagctc accgtggaca agagcaggtg gcagcagggg 600
aacgtcttct catgctccgt gatgcatgag gctctgcaca accactacac gcagaagagc 660
ctctccctgt ctccgggt 678
<210> 8
<211> 226
<212> PRT
<213> Homo sapiens
<400> 8
Asp Lys Thr His Thr Cys Pro Pro Cys Pro Ala Pro Glu Leu Leu Gly
1 5 10 15
Gly Pro Ser Val Phe Leu Phe Pro Pro Lys Pro Lys Asp Thr Leu Met
                        20 25 30
Ile Ser Arg Thr Pro Glu Val Thr Cys Val Val Val Asp Val Ser His
                35 40 45
Glu Asp Pro Glu Val Lys Phe Asn Trp Tyr Val Asp Gly Val Glu Val
        50 55 60
His Asn Ala Lys Thr Lys Pro Arg Glu Glu Gln Tyr Asn Ser Thr Tyr
65 70 75 80
Arg Val Val Ser Val Leu Thr Val Leu His Gln Asp Trp Leu Asn Gly
                                85 90 95
Lys Glu Tyr Lys Cys Lys Val Ser Asn Lys Ala Leu Pro Ala Pro Ile
                        100 105 110
Glu Lys Thr Ile Ser Lys Ala Lys Gly Gln Pro Arg Glu Pro Gln Val
                115 120 125
Tyr Thr Leu Pro Pro Ser Arg Asp Glu Leu Thr Lys Asn Gln Val Ser
        130 135 140
Leu Thr Cys Leu Val Lys Gly Phe Tyr Pro Ser Asp Ile Ala Val Glu
145 150 155 160
Trp Glu Ser Asn Gly Gln Pro Glu Asn Asn Tyr Lys Thr Thr Pro Pro
                              165 170 175
Val Leu Asp Ser Asp Gly Ser Phe Phe Leu Tyr Ser Lys Leu Thr Val
                        180 185 190
Asp Lys Ser Arg Trp Gln Gln Gly Asn Val Phe Ser Cys Ser Val Met
                195 200 205
His Glu Ala Leu His Asn His Tyr Thr Gln Lys Ser Leu Ser Leu Ser
        210 215 220
Pro Gly
225
<210> 9
<211> 66
<212> DNA
<213> Artificial
<220>
<223> Synthetic
<400> 9
ggtggctctg gagggggttc cggtggcgga ggttcaggtg goggaggggg ttcgggtggc 60
ggaggg 66
<210> 10
<211> 22
<212> PRT
<213> Artificial
<220>
<223> Synthetic
<400> 10
Gly Gly Ser Gly Gly Gly Ser Gly Gly Gly Gly Ser Gly Gly Gly Gly
1 5 10 15
Gly Ser Gly Gly Gly Gly
                        20
<210> 11
<211> 1974
<212> DNA
<213> Artificial
<220>
<223 > Synthetic
<400> 11
agtgataccg gtagaccttt cgtagagatg tacagtgaaa tccccgaaat tatacacatg 60
actgaaggaa gggagctcgt cattccctgc cgggttacgt cacctaacat cactgttact 120
ttaaaaaagt ttccacttga cactttgatc cctgatggaa aacgcataat ctgggacagt 180
agaaagggct tcatcatatc aaatgcaacg tacaaagaaa tagggcttct gacctgtgaa 240
gcaacagtca atgggcattt gtataagaca aactatctca cacatcgaca aaccaataca 300
atcatagatg tggttctgag tccgtctcat ggaattgaac tatctgttgg agaaaagctt 360
gtcttaaatt gtacagcaag aactgaacta aatgtgggga ttgacttcaa ctgggaatac 420
ccttcttcga agcatcagca taagaaactt gtaaaccgag acctaaaaac ccagtctggg 480
agtgagatga agaaattttt gagcacctta actatagatg gtgtaacccg gagtgaccaa 540
ggattgtaca cctgtgcagc atccagtggg ctgatgacca agaagaacag cacatttgtc 600
agggtccatg aaaagtactc catgaccccc ccgaccttga acatcacgga ggagtcacac 660
gtcatcgaca ccggtgacag cctgtccatc tcctgcaggg gacagcaccc cctcgagtgg 720
gcttggccag gagctcagga ggogccagcc accggagaca aggacagcga ggacacgggg 780
gtggtgcgag actgcgaggg cacagacgcc aggccctact gcaaggtgtt gctgctgcac 840
gaggtacatg ccaacgacac aggcagctac gtctgctact acaagtacat caaggcacgc 900
atcgagggca ccacggccgc cagctcctac gtgttcgtga gagactttga gcagccattc 960
atcaacaagc ctgacacgct cttggtcaac aggaaggacg ccatgtgggt gccctgtctg 1020
gtgtccatcc ccggcctcaa tgtcacgctg cgctcgcaaa gctcggtgct gtggccagac 1080
gggcaggagg tggtgtggga tgaccggcgg ggcatgctcg tgtccacgcc actgctgcac 1140
gatgccctgt acctgcagtg cgagaccacc tggggagacc aggacttcct ttccaacccc 1200
ttcctggtgc acatcacagg caacgagctc ggtggctctg gagggggttc cggtggcgga 1260
ggttcaggtg gcggaggggg ttcgggtggc ggaggggaca aaactcacac atgcccaccg 1320
tgcccagcac ctgaactcct ggggggaccg tcagtcttcc tcttcccccc aaaacccaag 1380
gacaccctca tgatctcccg gacccctgag gtcacatgcg tggtggtgga cgtgagccac 1440
gaagaccctg aggtcaagtt caactggtac gtggacggcg tggaggtgca taatgccaag 1500
acaaagccgc gggaggagca gtacaacagc acgtaccgtg tggtcagcgt cctcaccgtc 1560
ctgcaccagg actggctgaa tggcaaggag tacaagtgca aggtctccaa caaagccctc 1620
ccagccccca togagaaaac catctccaaa gccaaagggc agccccgaga accacaggtg 1680
tacaccctgc ccccatcccg ggatgagctg accaagaacc aggtcagcct gacctgcctg 1740
gtcaaaggct tctatcccag cgacatcgcc gtggagtggg agagcaatgg gcagccggag 1800
aacaactaca agaccacgcc tcccgtgctg gactccgacg gctccttctt cctctacagc 1860
aagctcaccg tggacaagag caggtggcag caggggaacg tcttctcatg ctccgtgatg 1920
catgaggctc tgcacaacca ctacacgcag aagagcctct ccctgtctcc gggt 1974
<210> 12
<211> 658
<212> PRT
<213> Artificial
<220>
<223> Synthetic
<400> 12
Ser Asp Thr Gly Arg Pro Phe Val Glu Met Tyr Ser Glu Ile Pro Glu
1 5 10 15
Ile Ile His Met Thr Glu Gly Arg Glu Leu Val Ile Pro Cys Arg Val
                        20 25 30
Thr Ser Pro Asn Ile Thr Val Thr Leu Lys Lys Phe Pro Leu Asp Thr
                35 40 45
Leu Ile Pro Asp Gly Lys Arg Ile Ile Trp Asp Ser Arg Lys Gly Phe
        50 55 60
Ile Ile Ser Asn Ala Thr Tyr Lys Glu Ile Gly Leu Leu Thr Cys Glu
65 70 75 80
Ala Thr Val Asn Gly His Leu Tyr Lys Thr Asn Tyr Leu Thr His Arg
                                85 90 95
Gln Thr Asn Thr Ile Ile Asp Val Val Leu Ser Pro Ser His Gly Ile
                        100 105 110
Glu Leu Ser Val Gly Glu Lys Leu Val Leu Asn Cys Thr Ala Arg Thr
                115 120 125
Glu Leu Asn Val Gly Ile Asp Phe Asn Trp Glu Tyr Pro Ser Ser Lys
        130 135 140
His Gln His Lys Lys Leu Val Asn Arg Asp Leu Lys Thr Gln Ser Gly
145 150 155 160
Ser Glu Met Lys Lys Phe Leu Ser Thr Leu Thr Ile Asp Gly Val Thr
                              165 170 175
Arg Ser Asp Gln Gly Leu Tyr Thr Cys Ala Ala Ser Ser Gly Leu Met
                        180 185 190
Thr Lys Lys Asn Ser Thr Phe Val Arg Val His Glu Lys Tyr Ser Met
                195 200 205
Thr Pro Pro Thr Leu Asn Ile Thr Glu Glu Ser His Val Ile Asp Thr
        210 215 220
Gly Asp Ser Leu Ser Ile Ser Cys Arg Gly Gln His Pro Leu Glu Trp
225 230 235 240
Ala Trp Pro Gly Ala Gln Glu Ala Pro Ala Thr Gly Asp Lys Asp Ser
                                245 250 255
Glu Asp Thr Gly Val Val Arg Asp Cys Glu Gly Thr Asp Ala Arg Pro
                        260 265 270
Tyr Cys Lys Val Leu Leu Leu His Glu Val His Ala Asn Asp Thr Gly
                275 280 285
Ser Tyr Val Cys Tyr Tyr Lys Tyr Ile Lys Ala Arg Ile Glu Gly Thr
        290 295 300
Thr Ala Ala Ser Ser Tyr Val Phe Val Arg Asp Phe Glu Gln Pro Phe
305 310 315 320
Ile Asn Lys Pro Asp Thr Leu Leu Val Asn Arg Lys Asp Ala Met Trp
                                325 330 335
Val Pro Cys Leu Val Ser Ile Pro Gly Leu Asn Val Thr Leu Arg Ser
                        340 345 350
Gln Ser Ser Val Leu Trp Pro Asp Gly Gln Glu Val Val Trp Asp Asp
                355 360 365
Arg Arg Gly Met Leu Val Ser Thr Pro Leu Leu His Asp Ala Leu Tyr
        370 375 380
Leu Gln Cys Glu Thr Thr Trp Gly Asp Gln Asp Phe Leu Ser Asn Pro
385 390 395 400
Phe Leu Val His Ile Thr Gly Asn Glu Leu Gly Gly Ser Gly Gly Gly
                                405 410 415
Ser Gly Gly Gly Gly Ser Gly Gly Gly Gly Gly Ser Gly Gly Gly Gly
                        420 425 430
Asp Lys Thr His Thr Cys Pro Pro Cys Pro Ala Pro Glu Leu Leu Gly
                435 440 445
Gly Pro Ser Val Phe Leu Phe Pro Pro Lys Pro Lys Asp Thr Leu Met
        450 455 460
Ile Ser Arg Thr Pro Glu Val Thr Cys Val Val Val Asp Val Ser His
465 470 475 480
Glu Asp Pro Glu Val Lys Phe Asn Trp Tyr Val Asp Gly Val Glu Val
                              485 490 495
His Asn Ala Lys Thr Lys Pro Arg Glu Glu Gln Tyr Asn Ser Thr Tyr
                        500 505 510
Arg Val Val Ser Val Leu Thr Val Leu His Gln Asp Trp Leu Asn Gly
                515 520 525
Lys Glu Tyr Lys Cys Lys Val Ser Asn Lys Ala Leu Pro Ala Pro Ile
        530 540 545
Glu Lys Thr Ile Ser Lys Ala Lys Gly Gln Pro Arg Glu Pro Gln Val
545 550 555 560
Tyr Thr Leu Pro Pro Ser Arg Asp Glu Leu Thr Lys Asn Gln Val Ser
                                565 570 575
Leu Thr Cys Leu Val Lys Gly Phe Tyr Pro Ser Asp Ile Ala Val Glu
                        580 585 590
Trp Glu Ser Asn Gly Gln Pro Glu Asn Asn Tyr Lys Thr Thr Pro Pro
                595 600 605
Val Leu Asp Ser Asp Gly Ser Phe Phe Leu Tyr Ser Lys Leu Thr Val
        610 615 620
Asp Lys Ser Arg Trp Gln Gln Gly Asn Val Phe Ser Cys Ser Val Met
625 630 635 640
His Glu Ala Leu His Asn His Tyr Thr Gln Lys Ser Leu Ser Leu Ser
                                645 650 655
Pro Gly
<210> 13
<211> 1363
<212> PRT
<213> Homo sapiens
<400> 13
Met Gln Arg Gly Ala Ala Leu Cys Leu Arg Leu Trp Leu Cys Leu Gly
1 5 10 15
Leu Leu Asp Gly Leu Val Ser Gly Tyr Ser Met Thr Pro Pro Thr Leu
                        20 25 30
Asn Ile Thr Glu Glu Ser His Val Ile Asp Thr Gly Asp Ser Leu Ser
                35 40 45
Ile Ser Cys Arg Gly Gln His Pro Leu Glu Trp Ala Trp Pro Gly Ala
        50 55 60
Gln Glu Ala Pro Ala Thr Gly Asp Lys Asp Ser Glu Asp Thr Gly Val
65 70 75 80
Val Arg Asp Cys Glu Gly Thr Asp Ala Arg Pro Tyr Cys Lys Val Leu
                                85 90 95
Leu Leu His Glu Val His Ala Asn Asp Thr Gly Ser Tyr Val Cys Tyr
                        100 105 110
Tyr Lys Tyr Ile Lys Ala Arg Ile Glu Gly Thr Thr Ala Ala Ser Ser
                115 120 125
Tyr Val Phe Val Arg Asp Phe Glu Gln Pro Phe Ile Asn Lys Pro Asp
        130 135 140
Thr Leu Leu Val Asn Arg Lys Asp Ala Met Trp Val Pro Cys Leu Val
145 150 155 160
Ser Ile Pro Gly Leu Asn Val Thr Leu Arg Ser Gln Ser Ser Val Leu
                              165 170 175
Trp Pro Asp Gly Gln Glu Val Val Trp Asp Asp Arg Arg Gly Met Leu
                        180 185 190
Val Ser Thr Pro Leu Leu His Asp Ala Leu Tyr Leu Gln Cys Glu Thr
                195 200 205
Thr Trp Gly Asp Gln Asp Phe Leu Ser Asn Pro Phe Leu Val His Ile
        210 215 220
Thr Gly Asn Glu Leu Tyr Asp Ile Gln Leu Leu Pro Arg Lys Ser Leu
225 230 235 240
Glu Leu Leu Val Gly Glu Lys Leu Val Leu Asn Cys Thr Val Trp Ala
                                245 250 255
Glu Phe Asn Ser Gly Val Thr Phe Asp Trp Asp Tyr Pro Gly Lys Gln
                        260 265 270
Ala Glu Arg Gly Lys Trp Val Pro Glu Arg Arg Ser Gln Gln Thr His
                275 280 285
Thr Glu Leu Ser Ser Ile Leu Thr Ile His Asn Val Ser Gln His Asp
        290 295 300
Leu Gly Ser Tyr Val Cys Lys Ala Asn Asn Gly Ile Gln Arg Phe Arg
305 310 315 320
Glu Ser Thr Glu Val Ile Val His Glu Asn Pro Phe Ile Ser Val Glu
                                325 330 335
Trp Leu Lys Gly Pro Ile Leu Glu Ala Thr Ala Gly Asp Glu Leu Val
                        340 345 350
Lys Leu Pro Val Lys Leu Ala Ala Tyr Pro Pro Pro Glu Phe Gln Trp
                355 360 365
Tyr Lys Asp Gly Lys Ala Leu Ser Gly Arg His Ser Pro His Ala Leu
        370 375 380
Val Leu Lys Glu Val Thr Glu Ala Ser Thr Gly Thr Tyr Thr Leu Ala
385 390 395 400
Leu Trp Asn Ser Ala Ala Gly Leu Arg Arg Asn Ile Ser Leu Glu Leu
                                405 410 415
Val Val Asn Val Pro Pro Gln Ile His Glu Lys Glu Ala Ser Ser Pro
                        420 425 430
Ser Ile Tyr Ser Arg His Ser Arg Gln Ala Leu Thr Cys Thr Ala Tyr
                435 440 445
Gly Val Pro Leu Pro Leu Ser Ile Gln Trp His Trp Arg Pro Trp Thr
        450 455 460
Pro Cys Lys Met Phe Ala Gln Arg Ser Leu Arg Arg Arg Gln Gln Gln
465 470 475 480
Asp Leu Met Pro Gln Cys Arg Asp Trp Arg Ala Val Thr Thr Gln Asp
                              485 490 495
Ala Val Asn Pro Ile Glu Ser Leu Asp Thr Trp Thr Glu Phe Val Glu
                        500 505 510
Gly Lys Asn Lys Thr Val Ser Lys Leu Val Ile Gln Asn Ala Asn Val
                515 520 525
Ser Ala Met Tyr Lys Cys Val Val Ser Asn Lys Val Gly Gln Asp Glu
        530 535 540
Arg Leu Ile Tyr Phe Tyr Val Thr Thr Ile Pro Asp Gly Phe Thr Ile
545 550 555 560
Glu Ser Lys Pro Ser Glu Glu Leu Leu Glu Gly Gln Pro Val Leu Leu
                                565 570 575
Ser Cys Gln Ala Asp Ser Tyr Lys Tyr Glu His Leu Arg Trp Tyr Arg
                        580 585 590
Leu Asn Leu Ser Thr Leu His Asp Ala His Gly Asn Pro Leu Leu Leu
                595 600 605
Asp Cys Lys Asn Val His Leu Phe Ala Thr Pro Leu Ala Ala Ser Leu
        610 615 620
Glu Glu Val Ala Pro Gly Ala Arg His Ala Thr Leu Ser Leu Ser Ile
625 630 635 640
Pro Arg Val Ala Pro Glu His Glu Gly His Tyr Val Cys Glu Val Gln
                                645 650 655
Asp Arg Arg Ser His Asp Lys His Cys His Lys Lys Tyr Leu Ser Val
                        660 665 670
Gln Ala Leu Glu Ala Pro Arg Leu Thr Gln Asn Leu Thr Asp Leu Leu
                675 680 685
Val Asn Val Ser Asp Ser Leu Glu Met Gln Cys Leu Val Ala Gly Ala
        690 695 700
His Ala Pro Ser Ile Val Trp Tyr Lys Asp Glu Arg Leu Leu Glu Glu
705 710 715 720
Lys Ser Gly Val Asp Leu Ala Asp Ser Asn Gln Lys Leu Ser Ile Gln
                                725 730 735
Arg Val Arg Glu Glu Asp Ala Gly Arg Tyr Leu Cys Ser Val Cys Asn
                        740 745 750
Ala Lys Gly Cys Val Asn Ser Ser Ala Ser Val Ala Val Glu Gly Ser
                755 760 765
Glu Asp Lys Gly Ser Met Glu Ile Val Ile Leu Val Gly Thr Gly Val
        770 775 780
Ile Ala Val Phe Phe Trp Val Leu Leu Leu Leu Ile Phe Cys Asn Met
785 790 795 800
Arg Arg Pro Ala His Ala Asp Ile Lys Thr Gly Tyr Leu Ser Ile Ile
                                805 810 815
Met Asp Pro Gly Glu Val Pro Leu Glu Glu Gln Cys Glu Tyr Leu Ser
                        820 825 830
Tyr Asp Ala Ser Gln Trp Glu Phe Pro Arg Glu Arg Leu His Leu Gly
                835 840 845
Arg Val Leu Gly Tyr Gly Ala Phe Gly Lys Val Val Glu Ala Ser Ala
        850 855 860
Phe Gly Ile His Lys Gly Ser Ser Cys Asp Thr Val Ala Val Lys Met
865 870 875 880
Leu Lys Glu Gly Ala Thr Ala Ser Glu His Arg Ala Leu Met Ser Glu
                                885 890 895
Leu Lys Ile Leu Ile His Ile Gly Asn His Leu Asn Val Val Asn Leu
                        900 905 910
Leu Gly Ala Cys Thr Lys Pro Gln Gly Pro Leu Met Val Ile Val Glu
                915 920 925
Phe Cys Lys Tyr Gly Asn Leu Ser Asn Phe Leu Arg Ala Lys Arg Asp
        930 935 940
Ala Phe Ser Pro Cys Ala Glu Lys Ser Pro Glu Gln Arg Gly Arg Phe
945 950 955 960
Arg Ala Met Val Glu Leu Ala Arg Leu Asp Arg Arg Arg Pro Gly Ser
                                965 970 975
Ser Asp Arg Val Leu Phe Ala Arg Phe Ser Lys Thr Glu Gly Gly Ala
                        980 985 990
Arg Arg Ala Ser Pro Asp Gln Glu Ala Glu Asp Leu Trp Leu Ser Pro
                995 1000 1005
Leu Thr Met Glu Asp Leu Val Cys Tyr Ser Phe Gln Val Ala Arg
        1010 1015 1020
Gly Met Glu Phe Leu Ala Ser Arg Lys Cys Ile His Arg Asp Leu
        1025 1030 1035
Ala Ala Arg Asn Ile Leu Leu Ser Glu Ser Asp Val Val Lys Ile
        1040 1045 1050
Cys Asp Phe Gly Leu Ala Arg Asp Ile Tyr Lys Asp Pro Asp Tyr
        1055 1060 1065
Val Arg Lys Gly Ser Ala Arg Leu Pro Leu Lys Trp Met Ala Pro
        1070 1075 1080
Glu Ser Ile Phe Asp Lys Val Tyr Thr Thr Gln Ser Asp Val Trp
        1085 1090 1095
Ser Phe Gly Val Leu Leu Trp Glu Ile Phe Ser Leu Gly Ala Ser
        1100 1105 1110
Pro Tyr Pro Gly Val Gln Ile Asn Glu Glu Phe Cys Gln Arg Leu
        1115 1120 1125
Arg Asp Gly Thr Arg Met Arg Ala Pro Glu Leu Ala Thr Pro Ala
        1130 1135 1140
Ile Arg Arg Ile Met Leu Asn Cys Trp Ser Gly Asp Pro Lys Ala
        1145 1150 1155
Arg Pro Ala Phe Ser Glu Leu Val Glu Ile Leu Gly Asp Leu Leu
        1160 1165 1170
Gln Gly Arg Gly Leu Gln Glu Glu Glu Glu Val Cys Met Ala Pro
        1175 1180 1185
Arg Ser Ser Gln Ser Ser Glu Glu Gly Ser Phe Ser Gln Val Ser
        1190 1195 1200
Thr Met Ala Leu His Ile Ala Gln Ala Asp Ala Glu Asp Ser Pro
        1205 1210 1215
Pro Ser Leu Gln Arg His Ser Leu Ala Ala Arg Tyr Tyr Asn Trp
        1220 1225 1230
Val Ser Phe Pro Gly Cys Leu Ala Arg Gly Ala Glu Thr Arg Gly
        1235 1240 1245
Ser Ser Arg Met Lys Thr Phe Glu Glu Phe Pro Met Thr Pro Thr
        1250 1255 1260
Thr Tyr Lys Gly Ser Val Asp Asn Gln Thr Asp Ser Gly Met Val
        1265 1270 1275
Leu Ala Ser Glu Glu Phe Glu Gln Ile Glu Ser Arg His Arg Gln
        1280 1285 1290
Glu Ser Gly Phe Ser Cys Lys Gly Pro Gly Gln Asn Val Ala Val
        1295 1300 1305
Thr Arg Ala His Pro Asp Ser Gln Gly Arg Arg Arg Arg Pro Glu
        1310 1315 1320
Arg Gly Ala Arg Gly Gly Gln Val Phe Tyr Asn Ser Glu Tyr Gly
        1325 1330 1335
Glu Leu Ser Glu Pro Ser Glu Glu Asp His Cys Ser Pro Ser Ala
        1340 1345 1350
Arg Val Thr Phe Phe Thr Asp Asn Ser Tyr
        1355 1360
<210> 14
<211> 103
<212> PRT
<213> Homo sapiens
<400> 14
Tyr Ser Met Thr Pro Pro Thr Leu Asn Ile Thr Glu Glu Ser His Val
1 5 10 15
Ile Asp Thr Gly Asp Ser Leu Ser Ile Ser Cys Arg Gly Gln His Pro
                        20 25 30
Leu Glu Trp Ala Trp Pro Gly Ala Gln Glu Ala Pro Ala Thr Gly Asp
                35 40 45
Lys Asp Ser Glu Asp Thr Gly Val Val Arg Asp Cys Glu Gly Thr Asp
        50 55 60
Ala Arg Pro Tyr Cys Lys Val Leu Leu Leu His Glu Val His Ala Asn
65 70 75 80
Asp Thr Gly Ser Tyr Val Cys Tyr Tyr Lys Tyr Ile Lys Ala Arg Ile
                                85 90 95
Glu Gly Thr Thr Ala Ala Ser
                        100
<210> 15
<211> 306
<212> PRT
<213> Homo sapiens
<400> 15
Tyr Ser Met Thr Pro Pro Thr Leu Asn Ile Thr Glu Glu Ser His Val
1 5 10 15
Ile Asp Thr Gly Asp Ser Leu Ser Ile Ser Cys Arg Gly Gln His Pro
                        20 25 30
Leu Glu Trp Ala Trp Pro Gly Ala Gln Glu Ala Pro Ala Thr Gly Asp
                35 40 45
Lys Asp Ser Glu Asp Thr Gly Val Val Arg Asp Cys Glu Gly Thr Asp
        50 55 60
Ala Arg Pro Tyr Cys Lys Val Leu Leu Leu His Glu Val His Ala Asn
65 70 75 80
Asp Thr Gly Ser Tyr Val Cys Tyr Tyr Lys Tyr Ile Lys Ala Arg Ile
                                85 90 95
Glu Gly Thr Thr Ala Ala Ser Ser Tyr Val Phe Val Arg Asp Phe Glu
                        100 105 110
Gln Pro Phe Ile Asn Lys Pro Asp Thr Leu Leu Val Asn Arg Lys Asp
                115 120 125
Ala Met Trp Val Pro Cys Leu Val Ser Ile Pro Gly Leu Asn Val Thr
        130 135 140
Leu Arg Ser Gln Ser Ser Val Leu Trp Pro Asp Gly Gln Glu Val Val
145 150 155 160
Trp Asp Asp Arg Arg Gly Met Leu Val Ser Thr Pro Leu Leu His Asp
                              165 170 175
Ala Leu Tyr Leu Gln Cys Glu Thr Thr Trp Gly Asp Gln Asp Phe Leu
                        180 185 190
Ser Asn Pro Phe Leu Val His Ile Thr Gly Asn Glu Leu Tyr Asp Ile
                195 200 205
Gln Leu Leu Pro Arg Lys Ser Leu Glu Leu Leu Val Gly Glu Lys Leu
        210 215 220
Val Leu Asn Cys Thr Val Trp Ala Glu Phe Asn Ser Gly Val Thr Phe
225 230 235 240
Asp Trp Asp Tyr Pro Gly Lys Gln Ala Glu Arg Gly Lys Trp Val Pro
                                245 250 255
Glu Arg Arg Ser Gln Gln Thr His Thr Glu Leu Ser Ser Ile Leu Thr
                        260 265 270
Ile His Asn Val Ser Gln His Asp Leu Gly Ser Tyr Val Cys Lys Ala
                275 280 285
Asn Asn Gly Ile Gln Arg Phe Arg Glu Ser Thr Glu Val Ile Val His
        290 295 300
Glu Asn
305
<210> 16
<211> 759
<212> PRT
<213> Artificial
<220>
<223> Fusion protein constructed from extracellular
domains of VEGFR-1, VEGFR-2, and VEGFR-3.
<400> 16
Ser Asp Thr Gly Arg Pro Phe Val Glu Met Tyr Ser Glu Ile Pro Glu
1 5 10 15
Ile Ile His Met Thr Glu Gly Arg Glu Leu Val Ile Pro Cys Arg Val
                        20 25 30
Thr Ser Pro Asn Ile Thr Val Thr Leu Lys Lys Phe Pro Leu Asp Thr
                35 40 45
Leu Ile Pro Asp Gly Lys Arg Ile Ile Trp Asp Ser Arg Lys Gly Phe
        50 55 60
Ile Ile Ser Asn Ala Thr Tyr Lys Glu Ile Gly Leu Leu Thr Cys Glu
65 70 75 80
Ala Thr Val Asn Gly His Leu Tyr Lys Thr Asn Tyr Leu Thr His Arg
                                85 90 95
Gln Thr Asn Thr Ile Ile Asp Val Val Leu Ser Pro Ser His Gly Ile
                        100 105 110
Glu Leu Ser Val Gly Glu Lys Leu Val Leu Asn Cys Thr Ala Arg Thr
                115 120 125
Glu Leu Asn Val Gly Ile Asp Phe Asn Trp Glu Tyr Pro Ser Ser Lys
        130 135 140
His Gln His Lys Lys Leu Val Asn Arg Asp Leu Lys Thr Gln Ser Gly
145 150 155 160
Ser Glu Met Lys Lys Phe Leu Ser Thr Leu Thr Ile Asp Gly Val Thr
                              165 170 175
Arg Ser Asp Gln Gly Leu Tyr Thr Cys Ala Ala Ser Ser Gly Leu Met
                        180 185 190
Thr Lys Lys Asn Ser Thr Phe Val Arg Val His Glu Lys Gly Gly Ser
                195 200 205
Gly Gly Gly Ser Gly Gly Gly Gly Ser Gly Gly Gly Gly Gly Ser Gly
        210 215 220
Gly Gly Gly Tyr Ser Met Thr Pro Pro Thr Leu Asn Ile Thr Glu Glu
225 230 235 240
Ser His Val Ile Asp Thr Gly Asp Ser Leu Ser Ile Ser Cys Arg Gly
                                245 250 255
Gln His Pro Leu Glu Trp Ala Trp Pro Gly Ala Gln Glu Ala Pro Ala
                        260 265 270
Thr Gly Asp Lys Asp Ser Glu Asp Thr Gly Val Val Arg Asp Cys Glu
                275 280 285
Gly Thr Asp Ala Arg Pro Tyr Cys Lys Val Leu Leu Leu His Glu Val
        290 295 300
His Ala Asn Asp Thr Gly Ser Tyr Val Cys Tyr Tyr Lys Tyr Ile Lys
305 310 315 320
Ala Arg Ile Glu Gly Thr Thr Ala Ala Ser Ser Tyr Val Phe Val Arg
                                325 330 335
Asp Phe Glu Gln Pro Phe Ile Asn Lys Pro Asp Thr Leu Leu Val Asn
                        340 345 350
Arg Lys Asp Ala Met Trp Val Pro Cys Leu Val Ser Ile Pro Gly Leu
                355 360 365
Asn Val Thr Leu Arg Ser Gln Ser Ser Val Leu Trp Pro Asp Gly Gln
        370 375 380
Glu Val Val Trp Asp Asp Arg Arg Gly Met Leu Val Ser Thr Pro Leu
385 390 395 400
Leu His Asp Ala Leu Tyr Leu Gln Cys Glu Thr Thr Trp Gly Asp Gln
                                405 410 415
Asp Phe Leu Ser Asn Pro Phe Leu Val His Ile Thr Gly Asn Glu Leu
                        420 425 430
Tyr Asp Ile Gln Leu Leu Pro Arg Lys Ser Leu Glu Leu Leu Val Gly
                435 440 445
Glu Lys Leu Val Leu Asn Cys Thr Val Trp Ala Glu Phe Asn Ser Gly
        450 455 460
Val Thr Phe Asp Trp Asp Tyr Pro Gly Lys Gln Ala Glu Arg Gly Lys
465 470 475 480
Trp Val Pro Glu Arg Arg Ser Gln Gln Thr His Thr Glu Leu Ser Ser
                              485 490 495
Ile Leu Thr Ile His Asn Val Ser Gln His Asp Leu Gly Ser Tyr Val
                        500 505 510
Cys Lys Ala Asn Asn Gly Ile Gln Arg Phe Arg Glu Ser Thr Glu Val
                515 520 525
Ile Val His Glu Asn Asp Lys Thr His Thr Cys Pro Pro Cys Pro Ala
        535 540 535
Pro Glu Leu Leu Gly Gly Pro Ser Val Phe Leu Phe Pro Pro Lys Pro
545 550 555 560
Lys Asp Thr Leu Met Ile Ser Arg Thr Pro Glu Val Thr Cys Val Val
                                565 570 575
Val Asp Val Ser His Glu Asp Pro Glu Val Lys Phe Asn Trp Tyr Val
                        580 585 590
Asp Gly Val Glu Val His Asn Ala Lys Thr Lys Pro Arg Glu Glu Gln
                595 600 605
Tyr Asn Ser Thr Tyr Arg Val Val Ser Val Leu Thr Val Leu His Gln
        610 615 620
Asp Trp Leu Asn Gly Lys Glu Tyr Lys Cys Lys Val Ser Asn Lys Ala
625 630 635 640
Leu Pro Ala Pro Ile Glu Lys Thr Ile Ser Lys Ala Lys Gly Gln Pro
                                645 650 655
Arg Glu Pro Gln Val Tyr Thr Leu Pro Pro Ser Arg Asp Glu Leu Thr
                        660 665 670
Lys Asn Gln Val Ser Leu Thr Cys Leu Val Lys Gly Phe Tyr Pro Ser
                675 680 685
Asp Ile Ala Val Glu Trp Glu Ser Asn Gly Gln Pro Glu Asn Asn Tyr
        690 695 700
Lys Thr Thr Pro Pro Val Leu Asp Ser Asp Gly Ser Phe Phe Leu Tyr
705 710 715 720
Ser Lys Leu Thr Val Asp Lys Ser Arg Trp Gln Gln Gly Asn Val Phe
                                725 730 735
Ser Cys Ser Val Met His Glu Ala Leu His Asn His Tyr Thr Gln Lys
                        740 745 750
Ser Leu Ser Leu Ser Pro Gly
                755
```

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A recombinant human Fc fusion protein, comprising an Ig-like domain of a first VEGF receptor, an Ig-like domain of a second VEGF receptor, and at least an Ig-like domain of a third VEGF receptor; wherein said third VEGF receptor comprises human VEGFR-3 (flt-4) having an amino acid sequence listed in SEQ ID NO: 13 with the proviso that the amino acids at positions 105 and 106 of SEQ ID NO: 13 are substitutable such that the amino acid sequence at positions 104-106 of SEQ ID NO: 13 is NDT, NDS, NXT, or NXS, wherein X is an amino acid selected from the group consisting of A, R, N, C, E, Q, G, H, I, L, K, M, F, P, S, T, W, Y, and V.

2. The fusion protein of claim 1; wherein said Ig-like domain of a first VEGF receptor comprises an amino acid sequence that is at least 95% identical to Ig-like domain 2 of human VEGFR-1 (flt-1); said Ig-like domain of a second VEGF receptor comprises an amino acid sequence that is at least 90% identical to Ig-like domain 3 of human VEGFR-2 (KDR); and said at least an Ig-like domain of a third VEGF receptor comprises an amino acid sequence that is at least 90% identical to Ig-like domains 1 and 2 of human VEGFR-3.

3. (canceled)

4. The fusion protein of claim 2; wherein said at least an Ig-like domain of a third VEGF receptor comprises an amino acid sequence that is at least 95% identical to Ig-like domains 1, 2, and 3 of human VEGFR-3.

5. The fusion protein of claim 2, further comprising (d) a multimerizing component linked to said Ig-like domains; wherein said multimerizing component comprises a portion of an Fc domain of human IgG1.

6. (canceled)

7. The fusion protein of claim 2; wherein said Ig-like domain 2 of human VEGFR-1 comprises an amino acid sequence listed in SEQ ID NO: 2; said Ig-like domain 3 of human VEGFR-2 comprises an amino acid sequence listed in SEQ ID NO: 4; said Ig-like domains 1 and 2 of human VEGFR-3 comprises an amino acid sequence listed in SEQ ID NO: 6; and said portion of an Fc domain of human IgG1 comprises an amino acid sequence listed in SEQ ID NO: 8.

8. A fusion protein comprising an amino acid sequence that is at least 95% identical to the amino acid sequence listed in SEQ ID NO: 12 with the proviso that the amino acids at positions 286 and 287 of SEQ ID NO: 12 are substitutable such that the amino acid sequence at positions 285-287 of SEQ ID NO: 12 is NDT, NDS, NXT, or NXS, wherein X is an amino acid selected from the group consisting of A, R, N, C, E, Q, G, H, I, L, K, M, F, P, S, T, W, Y, and V.

9. (canceled)

10. The fusion protein of claim 8, comprising an amino acid sequence as listed in SEQ ID NO:12.

11. The fusion protein of claim 10; wherein said fusion polypeptide binds to a VEGF family member with dissociation constant K_{d}≤10⁻⁹ M.

12. (canceled)

13. An isolated nucleic acid molecule encoding a fusion polypeptide of claim 1.

14. The isolated nucleic acid molecule of claim 13, having a sequence substantially as listed in SEQ ID NO: 11.

15. A vector comprising the nucleic acid molecule of claim 13.

16. The vector of claim 15, comprising said nucleic molecule operatively linked to an expression control sequence.

17. A host-vector system comprising the expression vector of claim 16 in a host cell.

18. A method of producing a substantially purified fusion polypeptide, which method comprises: (a) growing cells of the host-vector system of claim 17 under conditions permitting production of the fusion polypeptide; and (b) recovering the fusion or chimeric polypeptide to produce a recovered fusion polypeptide; and (c) purifying said recovered fusion polypeptide to produce the substantially purified fusion polypeptide.

19. A method for treating or controlling at least a disease, condition, or disorder, in a subject in need thereof, which has etiology in aberrant angiogenesis; said method comprising administering to said subject an amount of a composition of a fusion protein comprising an amino acid sequence that is at least 95% identical to the amino acid sequence listed in SEQ ID NO: 12 or SEQ ID NO: 16 with the proviso that the amino acids at positions 286 and 287 of SEQ ID NO: 12 or SEQ ID NO: 16 are substitutable such that the amino acid sequence at positions 285-287 of SEQ ID NO: 12 or SEQ ID NO: 16 is NDT, NDS, NXT, or NXS, wherein X is an amino acid selected from the group consisting of A, R, N, C, E, Q, G, H, I, L, K, M, F, P, S, T, W, Y, and V.

20. (canceled)

21. The method of claim 19; wherein said fusion protein has an amino acid sequence as listed in SEQ ID NO: 12 or SEQ ID NO: 16.

22. The method of claim 21; wherein said disease, condition, or disorder is selected from the group consisting of choroidal neovascularization, polypoidal choroidal vasculopathy, myopic neovascularization, retinal neovascularization, retinopathy of prematurity, vascular leak, retinal edema, diabetic macular edema, macular edema caused by retinal vein occlusion or non-infectious posterior uveitis, diabetic retinopathy, proliferative diabetic retinopathy, corneal neovascularization, and neovascular glaucoma.

23. The method of claim 22; wherein the subject is administered with a dose of about 25-4000 micrograms of the fusion polypeptide.

24. The method of claim 23; wherein the composition is administered to the subject as an eye drop, an intravitreal, subretinal, or suprachoroidal injection.

25. (canceled)

26. (canceled)

27. The method of claim 21; wherein said disease, condition, or disorder is a non-ocular disease, condition, or disorder that has etiology in aberrant angiogenesis.

28. The method of claim 27; wherein said disease, condition, or disorder is selected from the group consisting of cancers, psoriasis, rheumatoid arthritis, and atherosclerosis.


SEQUENCE LISTING (16 sequences)

SEQ ID NO: 1 - DNA, 306 bp, Homo sapiens
agtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcata

SEQ ID NO: 2 - protein, 102 aa, Homo sapiens
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTII

SEQ ID NO: 3 - DNA, 309 bp, Homo sapiens
gatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaag

SEQ ID NO: 4 - protein, 103 aa, Homo sapiens
DVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 5 - DNA, 614 bp, Homo sapiens
tactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagct

SEQ ID NO: 6 - protein, 205 aa, Homo sapiens
YSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNEL

SEQ ID NO: 7 - DNA, 678 bp, Homo sapiens
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 8 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - DNA, 66 bp, Artificial
  Synthetic
ggtggctctggagggggttccggtggcggaggttcaggtggcggagggggttcgggtggcggaggg

SEQ ID NO: 10 - protein, 22 aa, Artificial
  Synthetic
GGSGGGSGGGGSGGGGGSGGGG

SEQ ID NO: 11 - DNA, 1974 bp, Artificial
  Synthetic
agtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaagtactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctcggtggctctggagggggttccggtggcggaggttcaggtggcggagggggttcgggtggcggaggggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 12 - protein, 658 aa, Artificial
  Synthetic
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELGGSGGGSGGGGSGGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 1363 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 14 - protein, 103 aa, Homo sapiens
YSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAAS

SEQ ID NO: 15 - protein, 306 aa, Homo sapiens
YSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHEN

SEQ ID NO: 16 - protein, 759 aa, Artificial
  Fusion protein constructed from extracellular domains of VEGFR-1, VEGFR-2, and VEGFR-3.
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGSGGGSGGGGSGGGGGSGGGGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG